SEQUENCE LISTING (27 sequences)

SEQ ID NO: 1 - protein, 449 aa, synthetic construct
AQKGAESIGSQKENDLNLEDSSKKSHQNAKQDLPAVTEDSVSLFNGNKIFVSKEKNSSGKYDLRATIDQVELKGTSDKNNGSGTLEGSKPDKSKVKLTVSADLNTVTLEAFDASNQKISSKVTKKQGSITEETLKANKLDSKKLTRSNGTTLEYSQITDADNATKAVETLKNSIKLEGSLVGGKTTVEIKEGTVTLKREIEKDGKVKVFLNDTAGSNKKTGKWEDSTSTLTISADSKKTKDLVFLTDSTLTITVNSKKTKDLVFTKEKTLTVSADSKKIKDFVFLTDGTITVQQYNTAGTSLEGSASEIKNLSELKNALKWWNLDSKLSSNKEQKNNNNVKEVSDSVQEDGLNDLYNNQEKQKSFTKNFGERKYEDLINPIEPIIPSESPKNKANIPNISIAHTEKKETKKENLIPSTNEEKEADAAIKYLEENILKNSKFSELIREWW

SEQ ID NO: 2 - protein, 486 aa, synthetic construct
SETFTNKLKEKHTDLGKEGVTKGAEELGKLFESVEVLSKAAKEMLANSVKELTSSEEFSTKLKDNHAQLGIQGVTKGVEELEKLSGSLESLSSEDFTKKLEGEHAQLGIENVTAAELEKLFKAVENLAKAAKEMAKLKGEHTDLGKEGVTKGADELEKLFESVKNLSKAAKEMLTNSKESEKFAGKLKNEHASLGKKDATKGAKELKDLSDSVESLVKASDDFTKKLQSSHAQLGVAGGATTADELEKLFKSVESLAKAAQDALANSVNELTSKKLKEKHTDLGKKDATAAELEKLFESVENLAKAAKEMLSNSNKAFTDKLKSSHAELGIANGAATKGAQELEKLFESVKNLSKAAQETLNNSVKESESFTSEKFTKKLSESHADIGIQALKTNPTKTKGAEELDKLFKAVENLSKAAKEMLANSSEDFTNKLKNGNAQLGLAALKTNGTNDKGAKELKDLSDSVESLVKAAQVMLTNSVKELTS

SEQ ID NO: 3 - protein, 23 aa, synthetic construct
MAHHHHHHVGTGSNDDDDKSPDP

SEQ ID NO: 4 - protein, 279 aa, Borreliella burgdorferi
NLDSKLSSNKEQKNNNNVKEVSDSVQEDGLNDLYNNQEKQKSFTKNFGERKYEDLINPIEPIIPSESPKNKANIPNISIAHTEKKETKKENLIPSTNEEKEADAAIKYLEENILKNSKFSELIREVRVIKDEYALIKADLYDVIGKINNKKTSLMENPKNNRDKINKLTQLLQNNLKIDSELEQLINMIDMAENEISSAAFFFDNAQKRLKESIIKRLESKNNRSYALKLSRQALSDARSALSNLESFASKRIEPMVRKEEIKELIKHAKTVLESLNKK

SEQ ID NO: 5 - protein, 279 aa, Borreliella burgdorferi
NLDSKLSSNKEQKNNNNVKEVSNSVQEDGLNDLYSNQEKQKSFTKNFGEWKYEDLINPIEPIIPSESPKNKANIPNISIVHTQKKEIKEEDLIPSTNEEKEADEAIKYLEENILQNSKFSELIREVRVLKDEYALINSDFYDVIEKIHNKKTSLMENYKNNRDKINKLTLLQNNLKINIELEQLINMIDIAENEIRSAAFFFDTAQKRLKESIIKRLESKNNRSYYALELSRQALSDARSALSSLESFAFKRAEPMVRKKKIKELIKHAKTVLESLNKK

SEQ ID NO: 6 - protein, 285 aa, Borreliella burgdorferi
NLNSKLSGNKEEQKNNNDIKEALNGVQENAINNLYGNKKEKKDFIKNSEKLKDKGLDVTTLPLEPVVAPSVESAVSLGESNNRIGIPTISIEHNQKKEIKEEDFFPSTEEEKQADKAIKDIENLIGESGFPELIENVCSLKHEYTLIRSDFYDVITKIQNKKISLMKNSHNNRNKIRELVQLQNNLKIGDELDKIMGCIDTAEQEIRSAAFFFDEAKESLKEGIIKRLEKSKNRAASQLSKKALNRAEDALRCLENYSSKKGEAIGRRSFIKEVVEQAKNALSKS

SEQ ID NO: 7 - protein, 292 aa, Borreliella burgdorferi
NLDSKLPNKEQKNNNDIKETLGSSVQENALNNLYGNQEEKKDFKNFEELKDESLIALAKSLASTRPTTVGNIESAVLPVGHVVSLETSANKVSIPTISIKHNQKKEIKKEDLSPSTKEEKKADKAIKDIENLIRDSGFPELIESMYSLKHEYTLIRNNFYDVITKIRNKKTSLIKNGRNNRDKIKELTQLQNNLKIVDELDEIMVHIDIAEQEIRSAAFFFNEAKEILKEGIIKRLESENKVASQLARQALNKVEDALKSLEASSSKRGLAMGRRRIIKELIENAKTVLSKS

SEQ ID NO: 8 - protein, 76 aa, synthetic construct
NLDSKLSSNKEQKNNNNVKEVSDSVQEDGLNDLYNNQEKQKSFTKNFGERKYEDLINPIEPIIPSESPKNKANIPN

SEQ ID NO: 9 - protein, 76 aa, synthetic construct
NLDSKLSSNKEQKNNNNVKEVSNSVQEDGLNDLYNNQEKQKSFTKNFGEWKYEDLINPIEPIIPSESPKNKANIPN

SEQ ID NO: 10 - protein, 256 aa, synthetic construct
KQNVSSLDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYVLEGTLTAEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALK

SEQ ID NO: 11 - protein, 42 aa, synthetic construct
STLTITVNSKKTKDLVFTKEKTLIVSADSKKIKDFKTVFLTD

SEQ ID NO: 12 - protein, 280 aa, synthetic construct
AQKGAESIGSQKENDLNLEDSSKKSHQNAKQDLPAVTEDSVSLFNGNKIFVSKEKNSSGKYDLRATIDQVELKGTSDKNNGSGTLEGSKPDKSKVKLTVSADLNTVTLEAFDASNQKISSKVTKKQGSITEETLKANKLDSKKLTRSNGTTLEYSQITDADNATKAVETLKNSIKLEGSLVGGKTTVEIKEGTVTLKREIEKDGKVKVFLNDTAGSNKKTGKWEDSTSTLTISADSKKTKDLVFLTDGTITVQQYNTAGTSLEGSASEIKNLSELKNALK

SEQ ID NO: 13 - protein, 227 aa, synthetic construct
AQKGAESIGSQKENDLNLEDSSKKSHQNAKQDLPAVTEDSVSLFNGNKIFVSKEKNSSGKYDLRATIDQVELKGTSDKNNGSGTLEGSKPDKSKVKLTVSADLNTVTLEAFDASNQKISSKVTKKQGSITEETLKANKLDSKKLTRSNGTTLEYSQITDADNATKAVETLKNSIKLEGSLVGGKTTVEIKEGTVTLKREIEKDGKVKVFLNDTAGSNKKTGKWEDST

SEQ ID NO: 14 - protein, 51 aa, synthetic construct
LTISADSKKIKDFVFLTDGTITVQQYNTAGTSLEGSASEIKNLSELKNALK

SEQ ID NO: 15 - protein, 322 aa, synthetic construct
AQKGAESIGSQKENDLNLEDSSKKSHQNAKQDLPAVTEDSVSLFNGNKIFVSKEKNSSGKYDLRATIDQVELKGTSDKNNGSGTLEGSKPDKSKVKLTVSADLNTVTLEAFDASNQKISSKVTKKQGSITEETLKANKLDSKKLTRSNGTTLEYSQITDADNATKAVETLKNSIKLEGSLVGGKTTVEIKEGTVTLKREIEKDGKVKVFLNDTAGSNKKTGKWEDSTSTLTISADSKKTKDLVFLTDSTLTITVNSKKTKDLVFTKEKTLIVSADSKKIKDFKTVFLTDGTITVQQYNTAGTSLEGSASEIKNLSELKNALK

SEQ ID NO: 16 - protein, 665 aa, synthetic construct
NLDSKLSSNKEQKNNNNVKEVSDSVQEDGLNDLYNNQEKQKSFTKNFGERKYEDLINPIEPIIPSESPKNKANIPNISIAHTEKKETKKENLIPSTNEEKEADAAIKYLEENILKNSKFSELIREWWSETFTNKLKEKHTDLGKEGVTKGAEELGKLFESVEVLSKAAKEMLANSVKELTSSEEFSTKLKDNHAQLGIQGVTKGVEELEKLSGSLESLSSEDFTKKLEGEHAQLGIENVTAAELEKLFKAVENLAKAAKEMAKLKGEHTDLGKEGVTKGADELEKLFESVKNLSKAAKEMLTNSKESEKFAGKLKNEHASLGKKDATKGAKELKDLSDSVESLVKASDDFTKKLQSSHAQLGVAGGATTADELEKLFKSVESLAKAAQDALANSVNELTSKKLKEKHTDLGKKDATAAELEKLFESVENLAKAAKEMLSNSNKAFTDKLKSSHAELGIANGAATKGAQELEKLFESVKNLSKAAQETLNNSVKESESFTSEKFTKKLSESHADIGIQALKTNPTKTKGAEELDKLFKAVENLSKAAKEMLANSSEDFTNKLKNGNAQLGLAALKTNGTNDKGAKELKDLSDSVESLVKAAQVMLTNSVKELTSWWKKTAAWNSGTSTLTITVNSKKTKDLVFTKENTITVQQYDSNGTKLEGSAV

SEQ ID NO: 17 - protein, 615 aa, synthetic construct
NLDSKLSSNKEQKNNNNVKEVSDSVQEDGLNDLYNNQEKQKSFTKNFGERKYEDLINPIEPIIPSESPKNKANIPNISIAHTEKKETKKENLIPSTNEEKEADAAIKYLEENILKNSKFSELIREWWSETFTNKLKEKHTDLGKEGVTKGAEELGKLFESVEVLSKAAKEMLANSVKELTSSEEFSTKLKDNHAQLGIQGVTKGVEELEKLSGSLESLSSEDFTKKLEGEHAQLGIENVTAAELEKLFKAVENLAKAAKEMAKLKGEHTDLGKEGVTKGADELEKLFESVKNLSKAAKEMLTNSKESEKFAGKLKNEHASLGKKDATKGAKELKDLSDSVESLVKASDDFTKKLQSSHAQLGVAGGATTADELEKLFKSVESLAKAAQDALANSVNELTSKKLKEKHTDLGKKDATAAELEKLFESVENLAKAAKEMLSNSNKAFTDKLKSSHAELGIANGAATKGAQELEKLFESVKNLSKAAQETLNNSVKESESFTSEKFTKKLSESHADIGIQALKTNPTKTKGAEELDKLFKAVENLSKAAKEMLANSSEDFTNKLKNGNAQLGLAALKTNGTNDKGAKELKDLSDSVESLVKAAQVMLTNSVKELTSWW

SEQ ID NO: 18 - protein, 742 aa, synthetic construct
NLDSKLSSNKEQKNNNNVKEVSDSVQEDGLNDLYNNQEKQKSFTKNFGERKYEDLINPIEPIIPSESPKNKANIPNISIAHTEKKETKKENLIPSTNEEKEADAAIKYLEENILKNSKFSELIREWWSETFTNKLKEKHTDLGKEGVTKGAEELGKLFESVEVLSKAAKEMLANSVKELTSSEEFSTKLKDNHAQLGIQGVTKGVEELEKLSGSLESLSSEDFTKKLEGEHAQLGIENVTAAELEKLFKAVENLAKAAKEMAKLKGEHTDLGKEGVTKGADELEKLFESVKNLSKAAKEMLTNSKESEKFAGKLKNEHASLGKKDATKGAKELKDLSDSVESLVKASDDFTKKLQSSHAQLGVAGGATTADELEKLFKSVESLAKAAQDALANSVNELTSKKLKEKHTDLGKKDATAAELEKLFESVENLAKAAKEMLSNSNKAFTDKLKSSHAELGIANGAATKGAQELEKLFESVKNLSKAAQETLNNSVKESESFTSEKFTKKLSESHADIGIQALKTNPTKTKGAEELDKLFKAVENLSKAAKEMLANSSEDFTNKLKNGNAQLGLAALKTNGTNDKGAKELKDLSDSVESLVKAAQVMLTNSVKELTSWWNLDSKLSSNKEQKNNNNVKEVSDSVQEDGLNDLYNNQEKQKSFTKNFGERKYEDLINPIEPIIPSESPKNKANIPNISIAHTEKKETKKENLIPSTNEEKEADAAIKYLEENILKNSKFSELIREWW

SEQ ID NO: 19 - protein, 454 aa, synthetic construct
SETFTNKLKEKHTDLGKEGVTKGAEELGKLFESVEVLSKAAKEMLANSVKELTSSEEFSTKLKDNHAQLGIQGVTKGVEELEKLSGSLESLSSEDFTKKLEGEHAQLGIENVTAAELEKLFKAVENLAKAAKEMAKLKGEHTDLGKEGVTKGADELEKLFESVKNLSKAAKEMLTNSKESEKFAGKLKNEHASLGKKDATKGAKELKDLSDSVESLVKASDDFTKKLQSSHAQLGVAGGATTADELEKLFKSVESLAKAAQDALANSVNELTSKKLKEKHTDLGKKDATAAELEKLFESVENLAKAAKEMLSNSNKAFTDKLKSSHAELGIANGAATKGAQELEKLFESVKNLSKAAQETLNNSVKESESFTSTLTITVNSKKTKDLVFTKEKTLIVSADSKKIKDFKTVFLTDSTLTISADSKKTKDLVFLTDNTLTVSADSKKIKDFVFLTD

SEQ ID NO: 20 - DNA, 1347 bp, synthetic construct
  source 1..1347: mol_type = other DNA
gcgcagaagggtgcggagagcatcggcagccaaaaagaaaacgacctgaacctggaggatagcagcaagaaaagccaccagaacgcgaagcaagacctgccggcggtgaccgaggatagcgttagcctgttcaacggtaacaagatctttgttagcaaggagaagaacagcagcggcaaatacgacctgcgtgcgaccattgatcaggtggagctgaagggtaccagcgacaaaaacaacggtagcggcaccctggaaggcagcaaaccggataagagcaaagtgaagctgaccgttagcgcggacctgaacaccgttaccctggaggcgtttgacgcgagcaaccagaagatcagcagcaaagtgaccaagaaacaaggtagcattaccgaggaaaccctgaaggcgaacaaactggacagcaagaaactgacccgtagcaacggcaccaccctggaatatagccaaatcaccgacgcggataacgcgaccaaggcggttgagaccctgaagaacagcattaaactggaaggtagcctggtgggtggcaaaaccaccgttgagatcaaagaaggtaccgtgaccctgaagcgtgagattgaaaaagacggcaaagtgaaggtttttctgaacgataccgcgggtagcaacaagaaaaccggcaagtgggaggacagcaccagcaccctgaccatcagcgcggacagcaagaaaaccaaagatctggttttcctgaccgacagcaccctgaccattaccgtgaacagcaagaaaaccaaggatctggtttttaccaaagaaaagaccctgaccgtgagcgcggatagcaagaaaatcaaagacttcgtttttctgaccgatggtaccattaccgtgcagcaatacaacaccgcgggtaccagcctggaaggcagcgcgagcgagatcaaaaacctgagcgagctgaagaacgcgctgaaatggtggaacctggacagcaagctgagcagcaacaaggaacagaaaaacaacaacaacgtgaaagaagttagcgatagcgtgcaagaggacggtctgaacgatctgtacaacaaccaggagaaacaaaagagcttcaccaagaactttggcgagcgtaaatatgaagacctgatcaacccgattgaaccgatcattccgagcgagagcccgaaaaacaaggcgaacatcccgaacatcagcattgcgcacaccgagaagaaagaaaccaagaaagagaacctgattccgagcaccaacgaggaaaaggaagcggacgcggcgatcaaatatctggaggaaaacattctgaagaacagcaaattcagcgagctgattcgtgaatggtgg

SEQ ID NO: 21 - protein, 191 aa, synthetic construct
NNSGKDGNTSANSADESVKGPNLTEISKKITDSNAVLLAVKEVEALLSSIDEIAAKAIGKKIHQNNGLDTENNHNGSLLAGAYAISTLIKQKLDGLKNEGLKEKIDAAKKCSETFTNKLKEKHTDLGKEGVTDADAKEAILKTNGTKTKGAEELGKLFESVEVLSKAAKEMLANSVKELTSPVVAESPKKP

SEQ ID NO: 22 - DNA, 1458 bp, synthetic construct
  source 1..1458: mol_type = other DNA
agcgagacctttaccaacaaactgaaggagaaacacaccgatctgggcaaggaaggcgttaccaagggtgcggaggaactgggcaagctgttcgagagcgtggaagttctgagcaaggcggcgaaagagatgctggcgaacagcgtgaaagaactgaccagcagcgaggaatttagcaccaagctgaaagacaaccacgcgcagctgggtatccaaggcgtgaccaagggtgttgaggaactggagaaactgagcggcagcctggaaagcctgagcagcgaggatttcaccaagaaactggagggtgaacacgcgcagctgggcattgagaacgtgaccgcggcggagctggaaaagctgtttaaagcggtggaaaacctggcgaaggcggcgaaagaaatggcgaagctgaaaggcgagcacaccgacttaggcaaggaaggcgttactaaaggcgcggatgagctggaaaaactgtttgagagcgtgaaaaacctgagcaaagcggcgaaggaaatgctgaccaacagcaaggagagcgaaaaatttgcgggcaagctgaaaaacgagcacgcgagcctgggtaagaaagacgcgaccaagggcgcgaaagagctgaaggacctgagcgatagcgtggaaagcctggttaaggcgagcgacgatttcaccaagaaattacagagcagccacgcgcaactgggtgttgcgggtggcgcgaccaccgcggacgaactggagaaactgtttaagagcgttgaaagcctggcgaaagcggcgcaggatgcgctggcgaatagcgttaacgagctgaccagcaagaaactgaaggaaaaacacaccgacctgggcaagaaagatgctaccgcggcggagttagagaaactgtttgagagcgtggaaaacctggcgaaagcggcgaaggagatgctgagcaacagcaacaaagcgtttaccgacaagctgaaaagcagccacgcggaactgggtatcgcgaacggtgcggcgaccaagggtgcgcaggagctggaaaaactgttcgagagcgttaagaatctgagcaaggcggcgcaagaaaccctgaacaacagcgtgaaggagagcgaaagcttcaccagcgagaaatttaccaagaaactgagcgaaagccacgcggacatcggtattcaggcgctgaagaccaacccgaccaagaccaaaggcgcggaggaactggataaactgttcaaggcggttgagaatctgagcaaagcggcgaaagaaatgctggcgaacagcagcgaagactttaccaacaagctgaaaaacggtaacgcgcagctgggtctggcggcgctgaaaaccaacggtaccaacgataaaggcgcgaaggagctgaaagatctgagcgacagcgttgagagcctggttaaggcggcgcaagtgatgctgaccaacagcgttaaagaactgaccagc

SEQ ID NO: 23 - protein, 297 aa, Borreliella burgdorferi
MRKSLFLYALLMGGLMSCNLDSKLSSNKEQKNNNNVKEVSDSVQEDGLNDLYNNQEKQKSFTKNFGERKYEDLINPIEPIIPSESPKNKANIPNISIAHTEKKETKKENLIPSTNEEKEADAAIKYLEENILKNSKFSELIREVRVIKDEYALIKADLYDVIGKINNKKTSLMENPKNNRDKINKLTQLLQNNLKIDSELEQLINMIDMAENEISSAAFFFDNAQKRLKESIIKRLESKNNRSYALKLSRQALSDARSALSNLESFASKRIEPMVRKEEIKELIKHAKTVLESLNKK

SEQ ID NO: 24 - protein, 297 aa, Borreliella burgdorferi
MRKSLFLYTLLMGGLMSCNLDSKLSSNKEQKNNNNVKEVSNSVQEDGLNDLYSNQEKQKSFTKNFGEWKYEDLINPIEPIIPSESPKNKANIPNISIVHTQKKEIKEEDLIPSTNEEKEADEAIKYLEENILQNSKFSELIREVRVLKDEYALINSDFYDVIEKIHNKKTSLMENYKNNRDKINKLTLLQNNLKINIELEQLINMIDIAENEIRSAAFFFDTAQKRLKESIIKRLESKNNRSYYALELSRQALSDARSALSSLESFAFKRAEPMVRKKKIKELIKHAKTVLESLNKK

SEQ ID NO: 25 - protein, 303 aa, Borreliella burgdorferi
MKKKMFLYTLLTIGLMSCNLNSKLSGNKEEQKNNNDIKEALNGVQENAINNLYGNKKEKKDFIKNSEKLKDKGLDVTTLPLEPVVAPSVESAVSLGESNNRIGIPTISIEHNQKKEIKEEDFFPSTEEEKQADKAIKDIENLIGESGFPELIENVCSLKHEYTLIRSDFYDVITKIQNKKISLMKNSHNNRNKIRELVQLQNNLKIGDELDKIMGCIDTAEQEIRSAAFFFDEAKESLKEGIIKRLEKSKNRAASQLSKKALNRAEDALRCLENYSSKKGEAIGRRSFIKEVVEQAKNALSKS

SEQ ID NO: 26 - protein, 313 aa, Borreliella burgdorferi
MFLCKKNLFLYTLLTIGLISCNLDSKLPNKEQKNNNDIKETLGSSVQENALNNLYGNQEEKKDFKNFEELKDESLIALAKSLASTRPTTVGNIESAVLPVGHVVSLETSANKVSIPTISIKHNQKKEIKKEDLSPSTKEEKKADKAIKDIENLIRDSGFPELIESMYSLKHEYTLIRNNFYDVITKIRNKKTSLIKNGRNNRDKIKELTQLQNNLKIVDELDEIMVHIDIAEQEIRSAAFFFNEAKEILKEGIIKRLESENKVASQLARQALNKVEDALKSLEASSSKRGLAMGRRRIIKELIENAKTVLSKS

SEQ ID NO: 27 - protein, 40 aa, synthetic construct
STLTISADSKKTKDLVFLTDNTLTVSADSKKIKDFVFLTD

(19) United States
(12) Patent Application Publication
(10) Pub. No.: US 2026/0345759 A1
(43) Pub. Date: Nov. 5, 2026

(54) NOVEL CHIMERIC MULTI-PROTEIN BASED RECOMBINANT VACCINE ANTIGENS FOR PREVENTION OF LYME DISEASE IN ANIMALS AND HUMANS

(71) Applicant: VIRGINIA COMMONWEALTH UNIVERSITY, Richmond, VA (US)

(72) Inventors: Richard T. MARCONI, Midlothian, VA (US); Nathaniel O'BIER, Richmond, VA (US)

(21) Appl. No.: 19/113,555

(22) PCT Filed: Oct. 3, 2023
(86) PCT No.: PCT/US2023/075794

§ 371 Date: Mar. 20, 2025

Related U.S. Application Data
(60) Provisional application No. 63/412,655, filed on Oct. 3, 2022.

(51) Int. Cl. A61K 39/02; A61K 39/00; A61K 39/116; A61K 39/39; A61P 31/04; C07K 14/20; C12N 1/20; C12N 15/70; C12R 1/19

(52) U.S. Cl. CPC A61K 39/0225; A61K 39/116; A61K 39/39; A61P 31/04; C07K 14/20; C12N 1/20; C12N 15/70; A61K 2039/552; A61K 2039/55588; A61K 2039/575; A61K 2039/70; C07K 2319/00; C12R 2001/19

(57) ABSTRACT

A vaccine formulation for humans or other mammals (including dogs, horses, and cats) is provided. The vaccine formulation includes two chimeric proteins designed to elicit antibodies that bind to several targets on the surface of Lyme disease spirochetes during their residence in ticks and in mammals, and act synergistically to kill the bacteria through both antibody-mediated complement dependent and complement-independent mechanisms.

### CROSS-REFERENCE TO RELATED APPLICATIONS

This application is a continuation of U.S. provisional patent application 63/412,655, filed Oct. 3, 2022.

### STATEMENT OF FEDERALLY SPONSORED RESEARCH AND DEVELOPMENT

This invention was made with government support under 1R01AI141801-01A1, awarded by the National Institutes of Health (NIH) National Institute of Allergy and Infectious Diseases (NIAID). The United States government has certain rights in the invention.

### SEQUENCE LISTING

This application includes as the Sequence Listing the complete contents of the accompanying xml file “Sequence.xml”, created Oct. 1, 2023, containing 34,251 bytes, hereby incorporated by reference.

### FIELD OF THE INVENTION

The disclosure generally relates to vaccines against Lyme disease. In particular, the invention provides a vaccine formulation for humans and other mammals, which comprises two chimeric proteins designed to elicit antibodies that bind to targets on the surface of Lyme disease spirochetes during their residence in ticks and in mammals and act synergistically to kill the bacteria through an antibody-mediated complement-dependent and complement independent mechanisms.

### BACKGROUND OF THE INVENTION

Lyme disease (LD), first described in the 1970's, is now the most common arthropod-borne disease of humans and companion animals in the Northern Hemisphere. Over 150 million people in the US and 400 million people in Europe live in established endemic regions for LD. LD is a complex illness with variable presentation caused by species of the genus Borreliella. In North America, B. burgdorferi is the dominant species associated with disease. We collectively refer to all species associated with LD as the LD spirochetes. If not diagnosed and treated early, the LD spirochetes will disseminate and establish a persistent and debilitating infection. At the present time, there are no LD vaccines available for use in humans. The need for a vaccine is pressing and is considered a high priority by the National Institutes of Health and the Center for Disease Control.

The human LD vaccine, LymeRix® was available between 1998 and 2002. LymeRix® consisted of a single Borreliella protein called outer surface protein A (OspA). Sales of LymeRix® plummeted shortly after its introduction, leading to its voluntary withdrawal from the market by the manufacturer in 2002. The scientific limitations of LymeRix® were many. Most notably, it failed to induce long-term protection, mandating the need for frequent booster vaccinations. The requirement for multiple boosters can be traced to the mechanism of action of the vaccine and the environment-specific production of Borreliella outer surface proteins. OspA is only produced by Borreliella when they reside within the midgut of Ixodes ticks. When ticks feed, the LD spirochetes become exposed to the blood meal, which turns off the production of OspA. As a result, OspA is only present on the surface of the LD spirochetes in the tick midgut. This raises the question, how did LymeRix® provide any protection? The answer is as follows. Individuals immunized and boosted with LymeRix® develop a robust antibody response to OspA. When an infected tick feeds on an immunized individual, blood passes from the individual into the tick's midgut. If the antibody titer to OspA is sufficiently high, preformed antibodies in the blood can bind to OspA on the cell surface and kill the bacteria in the tick through an antibody-dependent complement-mediated mechanism. However, protective OspA antibody levels wane within 6 months of vaccination. When the antibody levels to OspA in the blood fall below a critical titer, some Borreliella cells are able to transmit into the vaccinated individual and establish an infection. Since the Borreliella are not producing OspA in mammals, they cannot be targeted by OspA antibodies in that environment. Equally important is the fact that a memory immune response does not develop in OspA-vaccinated individuals because there is no OspA present on the bacterial cell surface to stimulate such a response. In spite of the shortcomings of “OspA” only vaccines, a modified form of OspA is currently in phase III clinical trials. The sole protein in that vaccine is a modified form of OspA called VLA15. VLA15 is a multivalent engineered protein that includes segments of OspA variants found in Borreliella species present in N. America and Europe. As with LymeRix®, the mechanism of action of VLA15 is to kill the LD bacteria within the tick.

At the present time, there are essentially no effective strategies in place for the prevention of tick-borne diseases and in particular, there is no commercially available vaccine for Lyme disease. The potential impact of a chimeritope Lyme disease vaccine on human and animal health cannot be overstated. If available, it would be a new and powerful tool that can be used to combat the growing problem of Lyme disease. It would serve to relieve the socioeconomic stress Lyme disease currently places on healthcare systems in the US, Canada, Europe, and Asia.

To do so, there is a need in the art to develop a vaccine that can target spirochetes in both ticks and mammals and kill the bacteria through both antibody mediated complement-dependent and complement-independent mechanisms. In addition, a vaccine that can elicit memory immune responses would be highly beneficial since it would reduce the number and frequency of required boosters.

### SUMMARY

Provided herein are vaccine antigens and a vaccine formulation that targets LD spirochetes within both ticks and mammals. The vaccine formulation comprises two unique custom-designed recombinant proteins (chimeritopes) that are comprised of antigenic regions derived from four different B. burgdorferi proteins: OspA, OspB, OspC, and FtlA. The vaccine antigens are designated herein as BAF and CE-BBB19 chimeritopes. “Chimeritopes” are novel recombinant proteins created in the laboratory that comprise epitopes and/or specific protein segments derived from multiple different proteins or protein variants, and usually some unrelated but useful sequences. An advantage of chimeritopes in general, which is well-documented in the literature, is that they can be designed to elicit antibodies that target the numerous species of Borreliella that cause Lyme disease in humans, companion animals, wildlife, and other mammals. Further, a vaccine that targets spirochetes in both ticks and mammals, kills through both complement-dependent and complement-independent immune mechanisms, and elicits memory immune responses, such as the vaccine disclosed herein, is even more beneficial because it increases vaccine efficacy and reduces the number and frequency of required boosters.

### BRIEF DESCRIPTION OF THE FIGURES

FIG. 1. Demonstration that FtlA and FtlB are widely distributed among Lyme disease isolates and that antibodies to each protein cross-react. Borreliella isolates (indicated by labeling across the top of the figure) were grown to the mid-log phase, and then the cells were harvested and prepped for SDS-PAGE in AnyKda SDS-PAGE gels. The fractionated cell lysates were transferred to membranes and screened with the antisera indicated to the left. MW markers are shown on the left (in kDa).

FIG. 2. ELISA analyses of the Ftl proteins. The immunoreactivity of recombinant FtlA (r-FtlA), r-FtlB, r-FtlC, and r-FtlD with antiserum raised against each protein was assessed by ELISA (top panel) and immunoblotting. (Bottom panel). BSA served as the negative-control immobilized protein in the ELISA analyses. The antisera used in the assays are indicated along the x-axis, and the y-axis indicates the absorbance measured at 405 nm. ELISAs were run in triplicate. The recombinant Ftl proteins were also screened using an immunoblot format with each antiserum (from left to right: anti-FtlA, anti-FtlB, anti-FtlC, and anti-FtlD). As a negative control, an immunoblot was screened with preimmune serum (not shown).

FIGS. 3A and B. Bactericidal Activity of FtlA and FtlB antibodies. Hyperimmune sera raised against FtlA, FtlB, FtlC, and FtlD were tested for bactericidal activity against B. burgdorferi B31 (A) and 297 (B) in the presence of complement activity-certified guinea pig serum (GPS) or heat-inactivated (HI) GPS. Preimmune rat serum (PI) with GPS and high titer anti-OspA antiserum with GPS served as the negative and positive controls, respectively, for bactericidal activity. Error bars show standard deviations. The percent killing upon treatment was calculated (y-axis).

FIGS. 4A and B. Localization of the immunodominant domain and bactericidal epitopes of FtlA. (A) The immunodominant domain of FtlA was identified by generating and screening recombinant full-length FtlA and three overlapping FtlA fragments (F1, F2, and F3). The proteins were screened with sera from confirmed B. burgdorferi Ab-positive client-owned dogs by ELISA (n=50). The horizontal lines indicate the positive-threshold cutoff. Significance was determined by one-way ANOVA with 95% CIs, comparing mean absorbance readings of FtlA F2 and F3 to FtlA F1 (P, 0.0001). (B) To determine if the N-terminal domain of FtlA harbors the epitopes that elicit bactericidal antibodies, the F1, F2, and F3 fragments were incubated with anti-FtlA antiserum prior to its addition to live cells. Percent killing was determined. Significance was determined by one-way ANOVA with 95% CIs, comparing each test condition to the results for anti-FtlA antiserum with GPS (P, 0.0001). Error bars show standard deviations. *, P, 0.05.

FIG. 5. Antibodies elicited by immunization with OspB are bactericidal. Anti-OspB antiserum generated in rats or mice was incubated with B. burgdorferi B31 in the presence of activity-certified complement from Guinea pigs (GPS) or heat-inactivated (HI) GPS. The potent antibody-mediated complement-dependent killing was observed. The y-axis indicates the percentage of killing.

FIG. 6. The OspB-A1A15 and C.A.B.v1 chimeric constructs do not elicit antibodies with significant bactericidal activity. Antiserum generated against each protein was incubated with B. burgdorferi B31 in the presence of activity-certified complement from Guinea pigs (GPS). Significant antibody-mediated complement-dependent or complement-independent killing was not observed.

FIG. 7. Structural organization of the BAF chimeric protein. The known structure of OspB with the modifications indicated is shown. The loop domain into which the A1 and A15 epitopes of OspA were inserted is indicated by the box on the left. The small box on the top indicates the C-terminal domain of OspB. The N-terminal domain of FtlA was joined to the construct at this site. Note that the location of introduced W residues is not indicated but can be found in the BAF amino acid sequence in the text.

FIG. 8. Antibodies to CE-BBB19 kill through both complement-dependent and complement-independent mechanisms. Antiscrum was generated against CE-BBB19 in rats and tested for bactericidal antibodies. Percent killing is indicated on the y-axis. PI=preimmune; GPS=guinea pig serum, HI-GPS=heat inactivated GPS.

FIG. 9. Coadministration of BAF and CE-BBB19 induces strong IgG antibody responses. Mice were immunized with BAF and CE-BBB19 in alum (two doses, two weeks apart), and then serum was collected and screened by ELISA for IgG antibodies to each protein. The numbers below each bar are arbitrary numbers assigned to track each mouse. Dark and light gray bars indicate the absorbance reading obtained with a 1:1000 dilution of serum with the BAF and CE-BBB19 chimeritopes, respectively.

FIG. 10. IgG isotyping of mice immunized with BAF and CE-BBB19. Isotyping was performed to determine the dominant IgG isotype elicited by immunization with BAF/CE-BBB19. IgG1 titers were high, indicating that the antibody elicited by vaccination is favorable for activating complement. The first bar and second bar graphs in each pair indicate results obtained with BAF and CE-BBB19, respectively. The numbers below each graph are animal ID numbers. Preimmune serum as the negative control.

FIG. 11. The BAF/CE-BBB19 vaccine formulation elicits antibodies to diverse OspC types. Recombinant OspC types (indicated below the figure) were generated, purified, and immobilized in ELISA plates. The immobilized proteins were screened using standard ELISA approaches with preimmune serum (left bar in each pair) and pooled serum from mice immunized with BAF/CE-BBB19. The results obtained by screening OspC types derived from North American and European Borreliella isolates are shown. The strong reactivity of the hyperimmune serum with diverse OspC types is indicative of a broad protective range.

FIG. 12. Immunization of rhesus macaques induces bactericidal antibodies that provide protection through synergistic antibody-mediated complement and complement-independent mechanisms. Serum from Rhesus macaques vaccinated with BAF/CE-BBB19 (adjuvanted with alum) was tested for bactericidal activity using the identical protocol described above. Active GPS or heat-inactivated (HI) GPS was included. As reported above for CE-BBB19, both complement-dependent and independent killing were observed. This important finding indicates that antibodies can kill through two synergistic mechanisms. The designations below each bar are animal ID designations. Animal ND22 was a non-vaccinated negative control. Killing activity is indicated on the y-axis.

### DETAILED DESCRIPTION

As noted above, Lyme disease (LD) was first described in the US in the late 1970's and is now the most common arthropod-borne disease of humans and companion animals in the US, Canada, Europe, and Asia. LD is a complex illness with variable presentation. Obvious pathognomonic Erythema migrans (EM) lesions are not always present or evident, and the early stages of infection are generally non-descript. As a result, a diagnosis based on clinical presentation is often difficult. If not diagnosed and treated early, the LD spirochetes disseminate and establish a persistent and debilitating infection that is characterized by neurologic, cardiac, and/or rheumatologic manifestations. In light of the challenges associated with the diagnosis and treatment of LD and the potentially serious consequences of long-term infection, prevention through vaccination is an attractive path forward.

The present disclosure describes vaccine development efforts using an approach referred to as chimeritope technology. Chimeritopes are novel recombinant proteins created in the laboratory that comprise epitopes and/or specific protein segments derived from multiple different proteins or protein variants. The advantage to chimeritopes is that they can be designed to elicit antibodies that target the numerous species of Borreliella that can cause Lyme disease in humans, companion animals, wildlife, and other mammals, all in a single or a few recombinant proteins. Furthermore, the chimeritopes described here also possess the unique and highly desirable feature of being able to kill the LD spirochetes through both antibody-mediated complement dependent and complement-independent mechanisms.

The present disclosure provides two unique custom-designed recombinant proteins (also referred to herein as chimeritopes, vaccine antigens, and vaccinogens) that include antigenic regions derived from five different B. burgdorferi proteins: OspA, OspB, OspC, FtlA, and FtlB. In addition, a novel two-protein vaccine formulation comprising both of the two recombinant proteins is provided, as are methods of its use to vaccinate mammals against LD. These unique proteins, when administered together, advantageously elicit antibodies that target multiple proteins presented on the surface of LD spirochetes during their residence both in ticks and in mammals, rather than only when the spirochete is within a tick.

In order to facilitate the understanding of the present invention, the following definitions are provided:

Antigen: a term used historically to designate an entity that is bound by an antibody and also to designate the entity that induces the production of the antibody. More current usage limits the meaning of antigen to that entity bound by an antibody, while the word “immunogen” is used for the entity that induces antibody production. Where an entity discussed herein is both immunogenic and antigenic, reference to it as either an immunogen or antigen will typically be made according to its intended utility. The terms “antigen”, “antigenic region” “immunogen” and “epitope” may be used interchangeably herein. As used herein, an antigen, immunogen or epitope is generally a portion of a protein (e.g., a peptide or polypeptide).
Antibody-dependent complement-mediated killing: the lysis or killing of a bacterial cell by antibody through a mechanism that requires, and is dependent on, active proteins of the complement system. Different subtypes of IgG are more efficient at complement fixation than others. IgG1 and IgG3 are best at complement fixation. Note—we show immunization with CE-BBB19+BAF induces a strong IgG1 response.
Antibody-dependent complement-independent killing: antibody killing of a cell not dependent on complement. This can occur when the binding of antibodies to the cell surface interferes with critical biological processes that are required for the cell to survive. In some cases, antibodies bound to the cell can cause pores to form, which results in osmolysis.
Linker sequences: short peptide sequences encoding functional units that may be engineered or otherwise added at the ends or within recombinant proteins, polypeptides, peptides of interest. Linker sequences may be used as “handles” for protein purification, as detectable signals of expression or binding to other proteins or macromolecules, to modulate tertiary structure, or enhance antigenicity. Examples of linker sequences include but are not limited to an amino acid spacer, an amino acid linker, a signal sequence, a stop transfer sequence, a transmembrane domain, and a protein purification ligand.
Tags: Recombinant protein sequences that can be added to the N- or C-terminus of a recombinant protein for the purpose of identification or for purifying the recombinant protein for subsequent uses. Examples of recombinant protein tags that may be useful in practicing the invention include but are not limited to glutathione-S-transferase (GST), poly-histidine, maltose binding protein (MBP), FLAG, V5, halo, myc, hemaglutinin (HA), S-tag, calmodulin, tag, streptavidin binding protein (SBP), Softag1™, Softag3™, Xpress tag, isopeptag, Spy Tag, biotin carboxyl carrier protein (BCCP), GFP, Nus-tag, strep-tag, thioredoxin tag, TC tag, and Ty tag. All such tags are well-known to those of ordinary skill in the art of recombinant protein production and may or may not be removed before using tag-specific cleavage protocols. For example, a hexa-histidine tag is typically removed by incubation of the protein with the enzyme enterokinase, which recognizes a specific amino acid motif. Cleavage of this motif releases the His tag from the recombinant protein.
Epitope: a specific chemical domain on an antigen that is recognized by a B-cell receptor and which can be bound by a secreted antibody. The term as used herein is interchangeable with “antigenic determinant.” An epitope may comprise a single, non-interrupted, contiguous chain of amino acids joined together by peptide bonds to form a peptide or polypeptide. Such an epitope can be described by its primary structure, i.e., the linear sequence of amino acids in the peptide chain. Epitope may also refer to conformational epitopes, which are comprised of at least some amino acids that are not part of an uninterrupted, linear sequence of amino acids but which are brought into proximity to other residues in the epitope by secondary, tertiary, and/or quaternary interactions of the protein. Residues in conformational epitopes may be located far from other resides in the epitope with respect to primary sequence but may be spatially located near other residues in the conformational epitope due to protein folding.
Chimeric or fusion peptide or polypeptide: a recombinant or synthetic peptide or polypeptide whose primary sequence comprises two or more amino acid sequences that do not occur together in a single molecule in nature. The two or more sequences may be, for example, a peptide (e.g., an epitope or antigenic region) and a linker sequence, or two or more peptides (which may be the same or different) which are cither contiguous or separated by a linker sequence, etc.
Original or native or wild-type sequence: The sequence of a peptide, polypeptide, protein or nucleic acid as found in nature.
Recombinant peptide, polypeptide, protein, or nucleic acid: A man-made, non-natural peptide, polypeptide, protein, or nucleic acid that has been produced and/or manipulated using molecular biology techniques such as cloning, polymerase chain reaction (PCR), etc.
Synthetic peptide, polypeptide, protein or nucleic acid: A peptide, polypeptide, protein or nucleic acid that has been produced using chemical synthesis procedures.
Techniques for recombinant techniques and procedures may be generally performed as known in the art and described in, for example: Sambrook et al., Molecular Cloning: A Laboratory Manual, 4th ed., Cold Spring Harbor Laboratory Press, Cold Spring Harbor, N.Y. 2012; Journals such as Current Protocols in Molecular Biology (a Wiley brand) and Current Protocols in Immunology (a Wiley brand); Real-Time PCR: Current Technology and Applications, edited by Julie Logan, Kirstin Edwards and Nick Saunders, 2009, Caister Academic Press, Norfolk, UK; the book series Methods in Molecular Biology (Springer), etc.
Identity, similarity, and structural similarity. As used herein, a sequence of a peptide, polypeptide, or protein is “similar” to a reference sequence if the amino acid sequence possesses a specified amount of identity compared to the reference sequence. The similarity of two sequences can be compared along their entire lengths by aligning the residues to optimize the number of identical amino acids; gaps in either or both sequences are permitted in making the alignment in order to optimize the number of identical amino acids, although the amino acids in each sequence must nonetheless remain in their proper order. A pair-wise comparison analysis of amino acid sequences can be carried out using the BESTFIT algorithm in the GCG package (version 10.2, Madison Wis.). Alternatively, polypeptides may be compared using the Blastp program of the BLAST 2 search algorithm, as described by Tatiana et al., (FEMS Microbiol Lett, 174, 247-250 (1999)), and available on the National Center for Biotechnology Information (NCBI) website. The default values for all BLAST 2 search parameters may be used, including matrix=BLOSUM62; open gap penalty=11, extension gap penalty=1, gap x_dropoff=50, expect=10, word size=3, and filter on.

In the comparison of two amino acid sequences, similarity may be referred to by e.g. “percent identity” or “percent similarity.” “Identity” refers to the presence of identical amino acids along the entire length of the sequences that are being compared. “Similarity” generally refers to the presence of not only identical amino acids but also the presence of conservative substitutions. A conservative substitution for an amino acid may be selected from other members of the class to which the amino acid belongs. For example, it is well-known that in some instances, an amino acid belonging to a grouping of amino acids having a particular size or characteristic (such as charge, hydrophobicity and hydrophilicity) can be substituted for another amino acid without altering the activity of a protein, particularly in regions of the protein that are not directly associated with biological activity. Nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine and glutamine. Positively charged (basic) amino acids include arginine, lysine and histidine. Negatively charged (acidic) amino acids include aspartic acid and glutamic acid. Conservative substitutions include, for example, Lys for Arg and vice versa to maintain a positive charge; Glu for Asp and vice versa to maintain a negative charge; Ser for Thr so that a free-OH is maintained; and Gln for Asn to maintain a free —NH₂.

### The Vaccine Antigens

Details of the development of the vaccine antigens are provided in the Examples section. Briefly, the first vaccine antigen includes OspB as the backbone. OspB is an outer surface protein that is specifically produced by Lyme disease spirochete during residence in the midgut of an unfed tick. Linear epitopes A #1 and A #15 from the OspA protein were selected and inserted into a surface-exposed antigenic domain of OspB to yield an “OspB-A #1-A #15-OspB” recombinant protein. In addition, immunodominant N-terminal fragments of B. burgdorferi proteins FtlA and FtlB, which are expressed during transmission and infection of mammals, were identified. These N-terminal fragments are nearly identical in primary sequence (the alignment is shown in the Examples section), and that of FtlA was selected for inclusion in the recombinant construct. Specifically, this sequence was inserted at the C-terminal end of the construct to yield a chimeric protein comprising OspB, OspA epitopes (A #1 and A #15) and the N-terminal domain of FtlA. The final product (OspB backbone, OspA epitopes (A #1 and A #15), and the N-terminal domain of FtlA) is informally designated herein as the “BAF” construct, and the chimeric gene and protein were designated as the baf gene and the BAF protein, respectively. Several tryptophan residues (W) were inserted into the construct to increase quantification accuracy. The sequence of BAF, where the introduced tryptophan residues are shown in bold and underlined, is as follows:

BAF construct amino acid sequence
(SEQ ID NO: 1)
AQKGAESIGSQKENDLNLEDSSKKSHQNAKQDLPAVTEDSVSLFNGNKI
FVSKEKNSSGKYDLRATIDQVELKGTSDKNNGSGTLEGSKPDKSKVKLT
VSADLNTVTLEAFDASNQKISSKVTKKQGSITEETLKANKLDSKKLTRS
NGTTLEYSQITDADNATKAVETLKNSIKLEGSLVGGKTTVEIKEGTVTL
KREIEKDGKVKVFLNDTAGSNKKIGKWEDSTSTLTISADSKKTKDLVFL
TDSTLTITVNSKKTKDLVFTKEKTLTVSADSKKIKDFVFLTDGTITVQQ
YNTAGTSLEGSASEIKNLSELKNALKWWNLDSKLSSNKEQKNNNNVKEV
SDSVQEDGLNDLYNNQEKQKSFTKNFGERKYEDLINPIEPIIPSESPKN
KANIPNISIAHTEKKETKKENLIPSTNEEKEADAAIKYLEENILKNSKF
SELIREWW

To further increase the ability of the vaccine to kill spirochetes that have been transferred from a tick to an animal, a second recombinant protein, CE-BBB19, was developed. The second protein is a modified variant of a chimeric epitope-based protein (chimeritope) comprised of different variants of two OspC epitopes, L5 and H5, that were identified through epitope mapping, as described in the Examples section.

The amino acid sequence of CE-BBB19 is presented below.

CE-BBB19 amino acid sequence:
(SEQ ID NO: 2)
SETFTNKLKEKHTDLGKEGVTKGAEELGKLFESVEVLSKAAKEMLANSV
KELTSSEEFSTKLKDNHAQLGIQGVTKGVEELEKLSGSLESLSSEDFTK
KLEGEHAQLGIENVTAAELEKLFKAVENLAKAAKEMAKLKGEHTDLGKE
GVTKGADELEKLFESVKNLSKAAKEMLTNSKESEKFAGKLKNEHASLGK
KDATKGAKELKDLSDSVESLVKASDDFTKKLQSSHAQLGVAGGATTADE
LEKLFKSVESLAKAAQDALANSVNELTSKKLKEKHTDLGKKDATAAELE
KLFESVENLAKAAKEMLSNSNKAFTDKLKSSHAELGIANGAATKGAQEL
EKLFESVKNLSKAAQETLNNSVKESESFTSEKFTKKLSESHADIGIQAL
KTNPTKTKGAEELDKLFKAVENLSKAAKEMLANSSEDFTNKLKNGNAQL
GLAALKTNGTNDKGAKELKDLSDSVESLVKAAQVMLINSVKELTS

Those of skill in the art will recognize that, while in some aspects of the invention, the amino acid sequences that are chosen for inclusion in vaccine formulations of the invention correspond exactly to the primary amino acid sequences of sequences SEQ ID NO: 1 and SEQ ID NO: 2, this need not always be the case. The amino acid sequences may be altered somewhat and still be suitable for use as described herein. For example, certain conservative amino acid substitutions may be made without having a deleterious effect on the ability of the recombinant proteins to elicit an immune response. Those of skill in the art will recognize the nature of such conservative substitutions, for example, the substitution of a positively charged amino acid for another positively charged amino acid (e.g., K for R or vice versa), substitution of a negatively charged amino acid for another negatively charged amino acid (e.g. D for E or vice versa); substitution of a hydrophobic amino acid for another hydrophobic amino acid (e.g. substitution of A, V, L, I, W, etc. for one another); etc. Recombinant proteins resulting from all such substitutions or alterations of the sequences of the recombinant proteins that are disclosed herein are encompassed by the present invention, as long as the resulting recombinant proteins still function to elicit a suitable immune response an antibody response, preferably a protective antibody response, in a subject to whom the vaccine formulation is administered.

In addition, the amino acid sequences of the recombinant proteins of the invention need not encompass a full-length sequence as disclosed herein. Certain small N- and/or C-terminal and/or internal deletions of amino acids (e.g., from about 1-10 amino acids, such as about 1, 2, 3, 4, 5, 6, 7, 8, 9, or 10) may be tolerated without decreasing the ability of the proteins to elicit the desired immune response. CE-BBB19 (SEQ ID #2), which is a linear and unstructured protein as determined using nuclear magnetic resonance techniques, harbors several imperfect repeats of the L5 and H5 epitope domains from 10 different OspC types. Hence, it was designed with some degree of inherent sequence redundancy. While the full L5 and H5 repeats in the sequence are imperfect, some share segments with perfect identity. Some instances of this are the 8 amino acid sequence, NSVKELTSF (bold), which is repeated twice; the 6 amino acid sequence, KAAKEM (italicized), which is repeated 5 times; the 4 amino acid sequence, HDTL (underlined), which is repeated 3 times; and the 3 amino acid sequence, KLF (underlined and bolded), which is repeated 7 times. Deletion of one or more of a given repeat sequence or pair of different repeat sequences may not attenuate the recombinant proteins' function to elicit a suitable immune response, preferably a protective antibody response, in a subject to whom the vaccine formulation is administered. This also applies in a similar manner to the recombinant chimeritope of SEQ ID NO: 1. It is noted that, in some aspects, when such deletions are made, the remaining contiguous amino acids of the primary sequence remain the same. All such forms of the recombinant sequences disclosed herein are encompassed herein.

SEQ ID#2 with selected repeated sequences
indicated.
(SEQ ID NO: 2)
SETFTNKLKEKHTDLGKEGVTKGAEELGKLFESVEVLSKAAKEMLANSV
KELTSSEEFSTKLKDNHAQLGIQGVTKGVEELEKLSGSLESLSSEDFTK
KLEGEHAQLGIENVTAAELEKLFKAVENLAKAAKEMAKLKGEHTDLGKE
GVTKGADELEKLFESVKNLSKAAKEMLTNSKESEKFAGKLKNEHASLGK
KDATKGAKELKDLSDSVESLVKASDDFTKKLQSSHAQLGVAGGATTADE
LEKLFKSVESLAKAAQDALANSVNELTSKKLKEKHTDLGKKDATAAELE
KLFESVENLAKAAKEMLSNSNKAFTDKLKSSHAELGIANGAATKGAQEL
EKLFESVKNLSKAAQETLNNSVKESESFTSEKFTKKLSESHADIGIQAL
KINPTKTKGAEELDKLFKAVENLSKAAKEMLANSSEDFTNKLKNGNAQL
GLAALKTNGTNDKGAKELKDLSDSVESLVKAAQVMLTNSVKELTS

Such changes to the primary sequence may be introduced for any of a variety of reasons, for example, to eliminate or introduce a protease cleavage site, to increase or decrease solubility, to promote or discourage intra- or inter-molecular interactions such as folding, ionic interactions, salt bridges, etc., which might otherwise interfere with the presentation and accessibility of the individual epitopes along the length of a protein. Proteins resulting from all such changes are encompassed by the present invention as long as the resulting recombinant protein functions to elicit an antibody response, preferably a protective antibody response, in a subject to whom the vaccine formulation is administered.

In general, such substituted or modified protein sequences will be at least about 50% identical or similar to the corresponding sequence in the recombinant protein disclosed herein, or about 60 to 70, or 70 to 80, or 80 to 90% identical to the disclosed sequences (including all integers within these ranges to 0.1 decimal places), or even about 95, 96, 97, 98 or 99% identical (including all decimal fractions between these values).

Such protein variations may occur in either SEQ ID NO: 1 or SEQ ID NO: 2, or both, as long as the vaccine composition that includes the two proteins maintains a suitable, useful level of activity, i.e., as long as the vaccine formulation elicits production of antibodies to the antigens/epitopes present in the proteins. In preferred aspects, the vaccine formulation elicits a protective immune response.

In some aspects, the individual fragments (e.g., peptide segments derived from different native or other chimeric proteins) or epitopes in a recombinant vaccinogen may be separated from one another by intervening sequences that are more or less neutral in character, i.e., they do not in and of themselves elicit an immune response to Borreliella. However, such sequences may elicit antibody production. Such sequences may or may not be present between the segments in a chimera. If present, they may, for example, serve to separate the segments and contribute to the steric isolation of the segments from each other. Alternatively, such sequences may be simply artifacts of recombinant processing procedures, e.g., cloning procedures. Such sequences are typically known as linker or spacer peptides, many examples of which are known to those of skill in the art. See, for example, Crasto, C. J. and J. A. Feng. 2000. LINKER: a program to generate linker sequences for fusion proteins. Protein Engineering 13 (5): 309-312, which is a reference that describes unstructured linkers. Structured (e.g., helical) sequence linkers may also be designed using, for example, existing sequences that are known to have that secondary structure or using basic known biochemical principles to design the linkers.

In addition, other elements may be present in the chimeric proteins, for example, leader sequences or sequences that “tag” the protein to facilitate purification or detection of the protein, examples of which include but are not limited to histidine (e.g., hexahistidine) tags, detection tags (e.g., S-tag, or Flag-tag), other antigenic amino acid sequences such as known T-cell epitope containing sequences and protein stabilizing motifs, etc. In addition, the chimeric proteins may be chemically modified, e.g., by amidation, sulfonylation, lipidation, or other techniques that are known to those of skill in the art, as long as the activity of the proteins to elicit a suitable antibody response is not vitiated.

Generally, the naturally occurring leader sequences of the native proteins on which the chimeritopes are based are not included in the chimera. However, chimeras in which one or more leader sequences are included are also encompassed.

It is noted that while modest changes in the vaccinogen sequences disclosed herein may be tolerated without decreasing their effectiveness, the organization (e.g., the linear primary sequence of amino acids) of the recombinant proteins that is necessary to elicit a suitable immune response, or even to successfully produce the proteins, is neither random nor obvious. The sequences were developed by painstaking experimentation, as described in the Examples section.

### Compositions

The present disclosure also provides compositions (vaccine formulations) for use in eliciting an immune response, preferably a protective immune response. The compositions may be utilized as vaccines to prevent or treat Borreliella infection, particularly when manifested as Lyme disease (Lyme borreliosis). However, non-vaccine compositions (e.g., for laboratory purposes such as storage, purification protocols, etc.) are also encompassed.

The vaccine compositions generally include both of the recombinant proteins disclosed herein (SEQ ID NO: 1 and SEQ ID NO: 2, or a variant of one or both of SEQ ID NO: 1 and/or SEQ ID NO: 2 as described herein), which have been isolated and substantially purified, together with a pharmacologically suitable carrier. However, compositions comprising only one of the proteins (SEQ ID NO: 1 or SEQ ID NO: 2 or a variant of one or both of SEQ ID NO: 1 and/or SEQ ID NO: 2 as described herein) are also encompassed. Such compositions may elicit an immune response.

The preparation of compositions for use as vaccines is well known to those of skill in the art. Typically, such compositions are prepared either as liquid solutions or suspensions; however, solid forms such as tablets, pills, powders, and the like are also contemplated. Solid forms suitable for solution in, or suspension in, liquids prior to administration may also be prepared. The preparation may also be emulsified. The active ingredients may be mixed with excipients, which are pharmaceutically acceptable and compatible with the active ingredients. Suitable excipients are, for example, water, saline, dextrose, glycerol, ethanol, and the like, or combinations thereof. In addition, the composition may contain minor amounts of auxiliary substances such as wetting or emulsifying agents, pH buffering agents, and the like. If it is desired to administer an oral form of the composition, various thickeners, flavorings, diluents, emulsifiers, dispersing aids or binders, and the like may be added. The composition of the present invention may contain any such additional ingredients so as to provide the composition in a form suitable for administration. The final amount of one or both recombinant proteins in the formulations may vary. However, in general, the amount in the formulations will be from about 0.01-99%, weight/volume.

The vaccine preparations disclosed herein may further comprise an adjuvant, suitable examples of which include but are not limited to Seppic, Quil A, Alhydrogel, etc. Other compounds that are used as adjuvants include but are not limited to: lipid nanoparticles, azalides, ‘MF59’ (a submicron oil-in-water emulsion of squalene, polysorbate 80, and sorbitan trioleate), 1H-imidazo[4,5-c]quinolin-4-amine, various saponins, substituted pyrimidines (e.g., see issued U.S. patent Ser. No. 11/266,738, the complete contents of which are hereby incorporated by reference herein), CpG based adjuvants delivered in combination with other adjuvants that may include various formulations of alum and the like as known in the art.

### Methods of Eliciting an Immune Response

The present invention provides methods of eliciting an immune response to Borreliella and/or to vaccinate against Borreliella infection in mammals. The methods involve administering, to a mammal, a therapeutically effective amount of a composition comprising at least one, and usually both of the recombinant proteins disclosed herein. Administration elicits or causes an immune response, preferably a protective immune response.

By “eliciting an immune response,” we mean that administration of the compositions comprising the recombinant proteins disclosed herein causes the synthesis of specific antibodies at high titer and/or immune cell proliferation, as measured, e.g., by ³H thymidine incorporation or by other known techniques. By “protective immune response,” we mean that the vaccine elicits an immune response that results in the protection of a vaccinated organism against challenge with Borreliella. The protective response either wholly or partially prevents or arrests the development of symptoms related to Borreliella infection in an individual that has been vaccinated with the preparation disclosed herein and is then later exposed to the bacteria, in comparison to a non-vaccinated (e.g., adjunct alone) control organism, in which or in whom disease progression is not prevented. In other words, one or more symptoms of LD are prevented in vaccinated individuals who are later exposed to the bacteria and who could otherwise develop LD but for having been vaccinated. In another aspect, administration of the vaccine wholly or partially prevents and/or arrests symptoms already present in an individual who already has LD. The present vaccine formulations advantageously provide protection by killing Borreliella bacteria through antibody-mediated complement-dependent and complement-independent mechanisms.

Typical symptoms which may be prevented or ameliorated (lessened) include fever, headache, fatigue, and a characteristic skin rash called erythema migrans. If left untreated, the infection can spread to joints, the heart, and the nervous system, and the ensuing symptoms can also be prevented or ameliorated (lessened) in a subject who has already contracted a Lyme disease infection. Alternatively, an infected person may also be asymptomatic and benefit from receiving the vaccine disclosed herein by preventing the emergence of future symptoms.

In some aspects, the mammal to whom the vaccine is administered is a human. However, those of skill in the art will recognize that other mammals exist for which such vaccinations would also be desirable, e.g., the preparations may also be used for veterinary purposes. Examples include but are not limited to companion “pets” such as dogs, cats, etc.; food source, work and recreational animals such as cattle, horses, oxen, sheep, pigs, goats, and the like; wild animals that are protected in zoos or preserves; or wild animals that serve as a reservoir of Borreliella (e.g., mice, deer, etc.).

The vaccine preparations may be administered by any of the many suitable means that are well known to those of skill in the art, including but not limited to injection, inhalation, orally, intranasally, ingestion of a food product, etc. In most aspects, the mode of administration is subcutaneous, intraperitoneal, or intramuscular. In addition, the compositions may be administered in conjunction with other treatment modalities, such as substances that boost the immune system, various anti-bacterial chemotherapeutic agents, antibiotics, and the like.

The amount of the two proteins in a dose of the vaccine is a therapeutically effective amount. A “therapeutically effective amount” refers to an amount that is sufficient to elicit an immune response to both of the recombinant proteins, preferably an antibody response, more preferably a protective immune response. In some aspects, the response is an antibody-mediated complement-dependent response. The therapeutic and preventative value can be further enhanced by synergism with complement-independent responses that the vaccines elicit.

The amount of each protein in a single dose of vaccine generally ranges from about 1 to about 90 μg, for example, from about 0.1 to 300 μg, such as from about 0.5 to about 200 μg, or from about 1 to about 100 μg, such as about 1, 5, 10, 20, 30, 40, 50, 60, 70, 80, 90 or 100 μg per dose. In some aspects, the dose ranges from about 1 to about 90 μg.

The vaccine compositions disclosed herein may be administered according to any suitable (effective) schedule. Because of the excellent immune response elicited by the compositions, the administration of “boosters” (e.g., repeat administration) is less stringent than with previous LD vaccines. For example, the vaccine may be administered once every 6 months, or once a year, or once every 2-5 years, or even once every ten years. Typically, a “prime-boost” strategy is used at least during the first year of use, in which the initial vaccine dose is followed by a second dose that serves to generate immune memory cells and boost the response to future exposure to the disease-causing agent, in this case, Borreliella species. A prime-boost strategy may include, for example, a first dose followed by a second dose, generally between about 3 to about 8 weeks after the first, and thereafter as needed to provide immune protection, for example, yearly, every 5 years, or every 10 years.

The present invention also encompasses antibodies to the epitopes and/or to the chimeric polypeptides disclosed herein. Such antibodies may be polyclonal, monoclonal, or chimeric and may be generated in any manner known to those of skill in the art. In a preferred embodiment of the invention, the antibodies are bactericidal (borreliacidal), i.e., exposure of Borreliella spirochetes to the antibodies causes the death of the spirochetes, both within a tick or within the body of a vaccinated mammal. Such antibodies may be used in a variety of ways, e.g., as detection reagents to diagnose prior exposure to Borreliella, as a reagent in a kit for the investigation of Borreliella, to treat Borreliella infections, etc.

Preferably, the antibody response is protective, i.e., prevents or lessens the development of symptoms of disease in a vaccinated subject that is later exposed to Borreliella, compared to an unvaccinated subject.

Infections caused by several species and/or strains of Borreliella are prevented by the administration of the compositions. For example, infection by B. burgdorferi, B, garinii, B. mayonii, B. afzelii, B. carolinesis, B. americanum, B. bavariensis, B. andersonii, B. japonica, B. spielmanii, B. sinica, B. kurtenbachii, B. baltazardii and/or B. venezuelensis is prevented or treated.

### Polynucleotides

The invention further provides nucleic acid sequences that encode the recombinant proteins disclosed herein. Such nucleic acids include DNA, RNA and hybrids thereof, and the like. Further, the invention comprehends vectors that contain or house such coding sequences. Examples of suitable vectors include but are not limited to plasmids, cosmids, viral-based vectors, expression vectors, etc. In a preferred embodiment, the vector is a plasmid expression vector. Exemplary DNA sequences that encode SEQ ID NO: 1 and SEQ ID NO: 2 are presented in the Examples section (SEQ ID NOS: 20 and 22, respectively. However, those of skill in the art will recognize that, due to the degeneracy (redundancy) of the genetic code, many other sequences may be generated that would also encode the proteins. All nucleic acid sequences capable of encoding SEQ ID NO: 1 and SEQ ID NO: 2, and/or variants thereof as described herein, are encompassed herein.

As used herein, the terms “polynucleotide” or “nucleic acid” refer to deoxyribonucleic acid (DNA), ribonucleic acid (RNA) and DNA/RNA hybrids. Polynucleotides may be single-stranded or double-stranded, are recombinant and may be synthetic, or isolated from an organism that produces them, for example, a genetically engineered bacterial, yeast, insect or mammalian. Polynucleotides include, but are not limited to: pre-messenger RNA (pre-mRNA), messenger RNA (mRNA), plus-strand RNA (RNA(+)), minus-strand RNA (RNA(−)), PCR amplified DNA, complementary DNA (cDNA), synthetic DNA, or recombinant DNA.

In some aspects, polynucleotides are codon-optimized for gene expression in E. coli or any other suitable expression cell (insect, mammalian, other bacteria). As used herein, the term “codon-optimized” refers to substituting codons in a polynucleotide encoding a polypeptide or protein in order to increase the expression, stability and/or activity of the polypeptide based on, e.g. codon biases between two or more organisms or genes or synthetically constructed bias tables, variation in the degree of codon bias within an organism, gene, or set of genes, systematic variation of codons including context, variation of codons according to their decoding tRNAs, variation of codons according to GC %, either overall or in one position of the triplet, variation in degree of similarity to a reference sequence, for example, a naturally occurring sequence, variation in the codon frequency cutoff, structural properties of mRNAs transcribed from the DNA sequence, systematic variation of codon sets for each amino acid, and/or isolated removal of spurious translation initiation sites.

In some aspects, the polynucleotides encode at least one, and usually only one, of the chimeric vaccine antigens (or a variant) as disclosed herein, although polynucleotides that encode both vaccinogens (e.g., in tandem) are also encompassed. Various illustrative embodiments of such polynucleotides include but are not limited to those set forth herein, and variants thereof.

As used herein, the terms “polynucleotide variant” and “variant” and the like refer to polynucleotides displaying substantial sequence identity with a reference polynucleotide sequence or polynucleotides that hybridize with a reference sequence under stringent conditions. These terms also encompass polynucleotides that are distinguished from a reference polynucleotide by the addition, deletion, substitution, or modification of at least one nucleotide. Accordingly, the terms “polynucleotide variant” and “variant” include polynucleotides in which one or more nucleotides have been added or deleted, or modified, or replaced with different nucleotides. In this regard, it is well understood in the art that certain alterations, inclusive of mutations, additions, deletions, and substitutions, can be made to a reference polynucleotide whereby the altered polynucleotide retains the biological function or activity of the reference polynucleotide. All such polynucleotides are encompassed herein.

The phrase “sequence identity” or, for example, comprising a “sequence 50% identical to,” as used herein, refers to the extent that sequences are identical on a nucleotide-by-nucleotide over a window of comparison, similar to that of amino acid “identity” discussed herein. Thus, a “percentage of sequence identity” may be calculated by comparing two optimally aligned sequences over the window of comparison, determining the number of positions at which the identical nucleic acid base (e.g., A, T, C, G, I) occurs in both sequences to yield the number of matched positions, dividing the number of matched positions by the total number of positions in the window of comparison (i.e., the window size), and multiplying the result by 100 to yield the percentage of sequence identity. Included are nucleotides and polypeptides comprising at least about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% (or even 100%) sequence identity compared to any of the sequences described herein.

Sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (including but not limited to GAP, BESTFIT, FASTA, and TFASTA) etc. or the BLAST family of programs as for example, disclosed in the GCG (Genetics Computer Group) product, the Wisconsin Package 11.0 (or later when available), a recognized industry standard for sequence analysis of nucleic acids and protein sequences; and Ausubel et al., Current Protocols in Molecular Biology, John Wiley & Sons Inc, 2012.

The polynucleotides encompassed herein may be present in a “nucleic acid cassette,” which may be (but is not necessarily) an “expression cassette.” The term “expression cassette,” as used herein, refers to genetic sequences which can express an RNA and, subsequently, a polypeptide. In one embodiment, the nucleic acid cassette contains at least one gene(s)-of interest, e.g., a polynucleotide(s) of interest that encodes at least one chimera as disclosed herein. In another embodiment, the nucleic acid cassette contains one or more expression control sequences, e.g., a promoter (e.g. constitutive or inducible), enhancer, poly(A) sequence, polyadenylation signals, additional restriction enzyme sites, multiple cloning sites, internal ribosomal entry sites (IRES), recombinase recognition sites (e.g., LoxP, FRT, and Att sites), termination codons, transcriptional termination signals, post-transcription response elements, and polynucleotides encoding self-cleaving polypeptides, epitope tags, and a gene(s) of interest, e.g., a polynucleotide(s) of interest.

In order to express a desired vaccine antigen, a cassette encoding the polypeptide can be inserted into an appropriate vector, such as an expression vector. The nucleic acid cassette is positionally and sequentially oriented within a vector such that the nucleic acid in the cassette can be transcribed into RNA. Generally, the cassette can be removed and inserted into a plasmid or viral vector as a single unit. Illustrative examples of vectors include, but are not limited to plasmids, autonomously replicating sequences, phagemids, cosmids, artificial chromosomes such as yeast artificial chromosome (YAC), various episomal vectors, bacterial artificial chromosome (BAC), or P1-derived artificial chromosome (PAC), bacteriophages such as lambda phage or M13 phage, and animal viruses. Exemplary viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40). Exemplary expression vectors include but are not limited to pClneo vectors (Promega) for expression in mammalian cells; pLenti4/V5-DEST™, pLenti6/V5-DEST™, and pLenti6.2/V5-GW/lacZ (Invitrogen) for lentivirus-mediated gene transfer and expression in mammalian cells. A wide range of vectors can be used, for example pET vectors which are widely used for expression in bacteria and which were used in the Examples described herein.

Within a vector, a polynucleotide sequence of interest is generally “operably linked” to other elements of the cassette or vector so as the intended function of all elements is possible. In some aspects, the phrase refers to a functional linkage between a nucleic acid expression control sequence (such as a promoter, and/or enhancer) and a second polynucleotide sequence, e.g., a polynucleotide-of-interest, wherein the expression control sequence directs transcription of the nucleic acid corresponding to the second sequence.

Exemplary expression control sequences suitable for use according to the present disclosure include but are not limited to: cytomegalovirus (CMV) immediate early promoter, viral simian virus 40 (SV40) (e.g., early or late), Moloney murine leukemia virus (MoMLV) LTR promoter, Rous sarcoma virus (RSV) LTR, herpes simplex virus (HSV) (thymidine kinase) promoter, H5, P7.5, and P11 promoters from vaccinia virus, short elongation factor 1-alpha (EF1a-short) promoter, long elongation factor 1-alpha (EF1a-long) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus (Irions et al., Nature Biotechnology 25, 1477-1482 (2007)), a Ubiquitin C promoter (UBC), a phosphoglycerate kinase-1 (PGK) promoter, a cytomegalovirus enhancer/chicken β-actin (CAG) promoter, a β-actin promoter and a myeloproliferative sarcoma virus enhancer, negative control region deleted, d1587rev primer-binding site substituted (MND) U3 promoter (Haas et al. Journal of Virology. 2003; 77(17): 9439-9450).

### Producing the Recombinant Vaccinogens

The vaccine antigens may be produced by any suitable method, many of which are known to those of skill in the art. For example, they may be chemically synthesized or produced using recombinant DNA technology (e.g., via a vector encoding a vaccine antigen) in bacterial cells, in cell culture (mammalian, yeast, or insect cells), in plants or plant cells, or by cell-free prokaryotic or eukaryotic-based expression systems, by other in vitro systems, etc. In some embodiments, the vaccine antigens are produced using an E. coli recombinant expression system. Thus, the disclosure also encompasses bacterial, mammalian, yeast, and/or insect host cells, which comprise at least one copy of a nucleotide sequence encoding a recombinant vaccine antigen disclosed herein. In some aspects, the host cell is a bacterial cell. In further aspects, the bacterial cell is an E. coli host that has been genetically engineered to contain and express at least one nucleic acid sequence disclosed herein in a manner that results in the production of the encoded recombinant vaccine antigen, such as via a vector located therein.

Methods of making the recombinant vaccine antigens are also encompassed herein. Generally, the methods include genetically engineering a host cell to contain and express a nucleic acid encoding at least one recombinant vaccine antigen disclosed herein, e.g., by introducing a suitable vector into the host cell. The host cell is genetically engineered so that at least one vaccine antigen is produced by the host cell, either within the host cell or excreted into the medium in which the host cell is grown. Methods of making recombinant proteins using various types of host cells are known in the art, e.g., by growing/cultivating the host cell in a medium compatible with growth and then i) harvesting and lysing the host cells to release the proteins or, for excreted proteins ii) harvesting the growth medium. Various known steps of concentrating, purifying, and analyzing the harvested proteins are followed to achieve a desired level of purity for use in the diagnostics disclosed herein, e.g., filtration, centrifugation, various column purification methods (e.g., affinity chromatography), and the like. Purity levels and identity may be determined, e.g., by gel electrophoresis, HPLC, sequencing, mass spectrometry, and/or any combination of these.

It is to be understood that this invention is not limited to particular embodiments described, as such may, of course, vary. It is also to be understood that the terminology used herein is for the purpose of describing particular embodiments only and is not intended to be limiting since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Representative illustrative methods and materials are herein described; methods and materials similar or equivalent to those described herein can also be used in the practice or testing of the present invention.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual dates of public availability and may need to be independently confirmed.

It is noted that, as used herein and in the appended claims, the singular forms “a,” “an,” and “the” include plural referents unless the context clearly dictates otherwise. It is further noted that the claims may be drafted to exclude any optional element. As such, this statement is intended to serve as support for the recitation in the claims of such exclusive terminology as “solely,” “only,” and the like in connection with the recitation of claim elements or use of a “negative” limitations, such as “wherein [a particular feature or element] is absent,” or “except for [a particular feature or element],” or “wherein [a particular feature or element] is not present (included, etc.).”

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The invention is further described by the following non-limiting examples which further illustrate the invention, and are not intended, nor should they be interpreted to, limit the scope of the invention.

### EXAMPLES

### Example 1. Identification of Candidate Tick Phase and Mammalian Phase Proteins for Use in the Construction of Multi-Protein, Multi-Valent Chimeric Vaccine Antigens

To design chimeric proteins that could target the LD spirochetes in a) the tick environment and or b) during infection of mammals, an extensive literature and database search was conducted. These analyses did not provide clear guidance for selecting the optimal vaccine candidate. Hence, we analyzed LD spirochete genome sequences and known Borreliella protein structures and selected 60 candidates to investigate. All strains were grown at 37 °C in Barbour-Stoenner-Kelly (BSK)-H complete media supplemented with 6% rabbit serum (Sigma). Growth was monitored daily using simple wet-mounts and dark-field microscopy. The sixty genes were either PCR amplified from B. burgdorferi B31 or synthesized with codon optimization and cloned into the pET45b expression vector. For genes that included a sequence predicted to encode a leader sequence, the leader was omitted from the construct. The gene-carrying plasmids were propagated in Escherichia coli NovaBlue DE3 cells, purified, and transformed into E. coli BL21/DE3 cells. Protein expression was induced by overnight autoinduction or with IPTG (1 mM). When IPTG was used, a 4-6 hour induction timeframe was used. The induced cells were lysed using a high-pressure homogenizer, and the cell lysate was fractionated into soluble or insoluble phases using standard methods. Recombinant proteins that fractionated with the soluble phase were purified using non-denaturing conditions via an N-terminal hexahistidine tag (the sequence of which is presented below) using nickel affinity chromatography (ÄKTA purifier). The elution buffer, which contained imidazole, was removed by dialysis into phosphate-buffered saline (PBS; pH 7.4) across a 6 to 8 kDa molecular weight cut-off membrane. To purify proteins from the insoluble phase, the insoluble fraction was resuspended in 8 M urea with 40 mM imidazole. Cellular debris was removed by centrifugation (15,500×g; 30 m; 4 °C), and the supernatants were loaded onto a Poly-Prep Chromatography column pre-equilibrated in 50 mM Tris-HCl, 300 mM NaCl, pH 8.0, with 8 M urea. The protein-bound resin was washed, and the proteins were eluted using 250 mM imidazole. Urea and imidazole were removed by stepwise dialysis into phosphate-buffered saline (PBS; pH 7.4) with decreasing concentrations of urea. Protein concentrations were determined using a BCA assay.

To identify proteins that are expressed during infection in mammals, the recombinant proteins were immobilized in the wells of ELISA plates and tested for reactivity with several hundred sera from infected humans, dogs, wild canids (Eastern coyotes), mice, raccoons, foxes, eastern black bears, and other wildlife. Prior to use, the sera were confirmed to be LD antibody positive by ELISA screening using VlsE as the immobilized antigen. VlsE is a verified and USDA-approved diagnostic antigen for LD. Among the most immunoreactive proteins were members of protein family 12 and OspC. Protein family 12 consists of 4 uncharacterized linear plasmid-encoded proteins. The ORF designations assigned to these proteins in the B. burgdorferi B31 genome were BBG01, BBK01, BBH37, and BBJ08. We have renamed the ORFs as FtlA, FtlB, FtlC and FtlD, respectively. The % amino acid identity or similarity among these proteins compared to FtlA is shown in Table 1.

TABLE 1
% amino acid identity or similarity compared to FtIA

| | FtIA | FtIB | FtIC | FtID |
|---|---|---|---|---|
| Protein | (34.3 (Da) | (34.6 kDa) | (34.3 kDa) | (34.6 kDa) |
| FtIA | | 89.6 | 52.5 | 53.8 |
| FtIB | 96.3 | | 55.1 | 55.8 |
| FtIC | 81.5 | 81.8 | | 68.4 |
| FtIC | 77.6 | 77.9 | 87.5 | |

The individual sequences for members of the Ftl proteins are provided below followed by an alignment of the complete sequences.

### SEQ ID NO: 3

Protein Name: His Tag Sequence with Enterokinase Cleavage Site.
NOTES: All recombinant proteins that were generated and purified below harbor this N-terminal his tag/enterokinase cleavage sequence. The isolated sequence is shown here. Note that the proteins can be produced using any N-terminal tag sequence.

Protein sequence:
(SEQ ID NO: 3)
MAHHHHHHVGTGSNDDDDKSPDP

### SEQ ID NO: 4

Protein Name: FtlA (BBK01) B. burgdorferi B31

### Protein ID: AAC66147.1

NOTES: Member of protein family 12. The leader peptide was omitted. The protein was readily expressed in E. coli as a soluble protein and was purified to homogeneity. As detailed below, the underlined sequence spanning residues 19-143 is the domain we identified that induces high titer, immunodominant, bactericidal antibodies.

(SEQ ID NO: 4)
NLDSKLSSNKEQKNNNNVKEVSDSVQEDGLNDLYNNQEKQKSFTKNFGE
RKYEDLINPIEPIIPSESPKNKANIPNISIAHTEKKETKKENLIPSTNE
EKEADAAIKYLEENILKNSKFSELIREVRVIKDEYALIKADLYDVIGKI
NNKKTSLMENPKNNRDKINKLTQLLQNNLKIDSELEQLINMIDMAENEI
SSAAFFFDNAQKRLKESIIKRLESKNNRSYALKLSRQALSDARSALSNL
ESFASKRIEPMVRKEEIKELIKHAKTVLESLNKK

### SEQ ID NO: 5

Protein Name: FtlB (BBG01) B. burgdorferi B31

### Protein ID: AAC66073.1

NOTES: Member of protein family 12. The leader peptide is omitted. The protein was readily expressed in E. coli as a soluble protein and was purified to homogeneity. The underlined sequence shares a high amino acid identity value with the N-terminal domain of FtlA shown in the previous sequence.

(SEQ ID NO: 5)
NLDSKLSSNKEQKNNNNVKEVSNSVQEDGLNDLYSNQEKQKSFTKNFGE
WKYEDLINPIEPIIPSESPKNKANIPNISIVHTQKKEIKEEDLIPSTNE
EKEADEAIKYLEENILQNSKFSELIREVRVLKDEYALINSDFYDVIEKI
HNKKTSLMENYKNNRDKINKLTLLQNNLKINIELEQLINMIDIAENEIR
SAAFFFDTAQKRLKESIIKRLESKNNRSYYALELSRQALSDARSALSSL
ESFAFKRAEPMVRKKKIKELIKHAKTVLESLNKK

### SEQ ID NO: 6

Protein Name: FLIC (BBH37) B. burgdorferi B31

### Protein ID: AAC66018.1

NOTES: In the sequence below, the leader peptide was omitted. The protein was readily expressed in E. coli as a soluble protein and was purified to homogeneity. While FtlC is classified as a member of protein family 12, we determined that the wild-type protein is not exported to the cell surface and that it oligomerizes. Oligomerization would interfere with the design of a chimeric vaccine antigen so the protein, or portions thereof, were not considered further. (10.1128/iai.00364-22). For this reason, FtlC was not incorporated into any of the chimeric constructs.

(SEQ ID NO: 6)
NLNSKLSGNKEEQKNNNDIKEALNGVQENAINNLYGNKKEKKDFIKNSE
KLKDKGLDVTTLPLEPVVAPSVESAVSLGESNNRIGIPTISIEHNQKKE
IKEEDFFPSTEEEKQADKAIKDIENLIGESGFPELIENVCSLKHEYTLI
RSDFYDVITKIQNKKISLMKNSHNNRNKIRELVQLQNNLKIGDELDKIM
GCIDTAEQEIRSAAFFFDEAKESLKEGIIKRLEKSKNRAASQLSKKALN
RAEDALRCLENYSSKKGEAIGRRSFIKEVVEQAKNALSKS

### SEQ ID NO: 7

Protein Name: FtlD (BBJ08) B. burgdorferi B31

### Protein ID: AAC66096.1

Notes: The leader peptide is omitted. The protein was readily expressed in E. coli as a soluble protein and was purified to homogeneity. Although FtlD when delivered to rats, was immunogenic, the anti-FtlD antibodies could not bind to other family 12 proteins. Based on this fact, coupled with the non-universal distribution of FtlD amongst isolates, it was not considered further for use in the chimeric constructs described below.

(SEQ ID NO: 7)
NLDSKLPNKEQKNNNDIKETLGSSVQENALNNLYGNQEEKKDFKNFEEL
KDESLIALAKSLASTRPTTVGNIESAVLPVGHVVSLETSANKVSIPTIS
IKHNQKKEIKKEDLSPSTKEEKKADKAIKDIENLIRDSGFPELIESMYS
LKHEYTLIRNNFYDVITKIRNKKTSLIKNGRNNRDKIKELTQLQNNLKI
VDELDEIMVHIDIAEQEIRSAAFFFNEAKEILKEGIIKRLESENKVASQ
LARQALNKVEDALKSLEASSSKRGLAMGRRRIIKELIENAKTVLSKS

To assess regions of identity in the family 12 proteins, an alignment was generated and is presented below, where each of the proteins is shown with a leader sequence having the following SEQ ID NOS: FtlA with leader (SEQ ID NO: 23), FtlB with leader (SEQ ID NO: 24). FtlC with leader (SEQ ID NO: 25) and FtlC with leader (SEQ ID NO: 26).

| | | |
|---|---|---|
| Ft1A | M---RKSLFLYALLMGGLMSCNLDSKLSSNKE-QKNNNNVKKV-SDSVQEDGLNDLYNNQ | 55 |
| Ft1B | M---RKSLFLYTLLMGGLMSCNLDSKLSSNKE-QKNNNNVKKV-SNSVQEDGLNDLYSNQ | 55 |
| Ft1C | M---KKKMFLYTLLTIGLMSCNLNSKLSGNKEEQKNNNDIKEALN-TVQENALNNLYGNK | 56 |
| Ft1D | MFLCKKNLFLYTLLTIGLISCNLDSKLP-NKE-QKNNNDIKETLGSSVQENALNNLYGNQ | 58 |
| | *:.:***:** **:****:*** *** *****::**. . .***:.:*:**.*: | |
| Ft1A | EKQKSFTKNFGERKYEDLIN---------------------PIEPIIPSESPKNKANIPN | 94 |
| Ft1B | EKQKEFTKNFGEWKYEDLIN---------------------PIEPIIPEESFKNKANIPN | 94 |
| Ft1C | KEKKDFIKNSEKLKDKGLDVTTLPL---EPV-------VAPSVESAVSLGESNNRIGIPT | 106 |
| Ft1D | EEKKDFKNFE-ELKDESLIALAKSLASTRPTTVGNIESAVLPVGHVVSLETSANKVSIPT | 117 |
| | :::*.* : : * :.* : : *: .**. | |
| Ft1A | ISIANTEKKETKKENLIPSTNEEKEADAAIKYLEENTLKNSKFSELIREVRVIKDEYALI | 154 |
| Ft1B | ISIVHTQKKEIKEEDLIPSTNEEKEADEAIKYLEENILQNSKFSELIREVRVLKDEYALI | 154 |
| Ft1C | ISIEHNQKKEIKEEDFFPSTEEEKQADKAIKDIE-NLIGESGFFELIENVCSLKHEYTLI | 165 |
| Ft1D | ISIKHNQKKEIKKEDLSFSTKEEKKADKAIKDIE-NLIRDSGFPELIESMYSLKHEYTLI | 176 |
| | *** *.:*** *:*:: ***:***:** *** :* *:: :* * ***..: :*.**:** | |
| Ft1A | KADLYDWIGKINNKKTSLMENPKNNRDKINKLTQLLQNNLKIDSELEQLIYNIDMAENEI | 215 |
| Ft1B | NSDFYDVIEKINNKKTSLMENYKNNRDKINKLT-LLQNNLKINIELEQLINMIDIAENEI | 213 |
| Ft1C | RSDFYDVITKIQNKKISLMKNSMNNRNKIRELVQ-LQNNLNIGDELDKIMGCIDTAEQEI | 224 |
| Ft1D | RNNFYDVITKIRNKKTSLIKNGRNNRDKIKELTQ-LQNNLKIVDELDEIMVHIDIAEQIE | 235 |
| | . ::**** **.*** **::* :***:**.:*. ******* **:::: ** **:** | |
| Ft1A | SSAAFFFDNAQKRLKESIIKRLSSKNNRS-YALKLSRQALSDARSALSNLESFASKRIEP | 273 |
| Ft1B | RSAAFFFDTAQKRLKKSIIKRLESKNNRSYYALELSRQALSDARSALSSLESFAFKRAEP | 273 |
| Ft1C | RSAAFFFDEAKESLKEGIIKRLEKSKNNA--ASQLSKKALNRAEDALRCLENYSSKKGEA | 282 |
| Ft1D | RSAAFFFNEAKEILKEGIIKRLES-ENKV--ASQLARQALNKVEDALKSLEASSSKRGLA | 292 |
| | ******: *:: ***.******. :*: * :*:::**. ...** ** : *: | |
| Ft1A | MVRKKEIKELIKHAKTVLESLNKK | 297 |
| Ft1B | MVRKKKIKELIKNAKTVLEELNKK | 297 |
| Ft1C | IGRRSTIKEVVEQAKNALSKS--- | 303 |
| Ft1D | MGRRRIIKELINNAKTVLSKS--- | 313 |

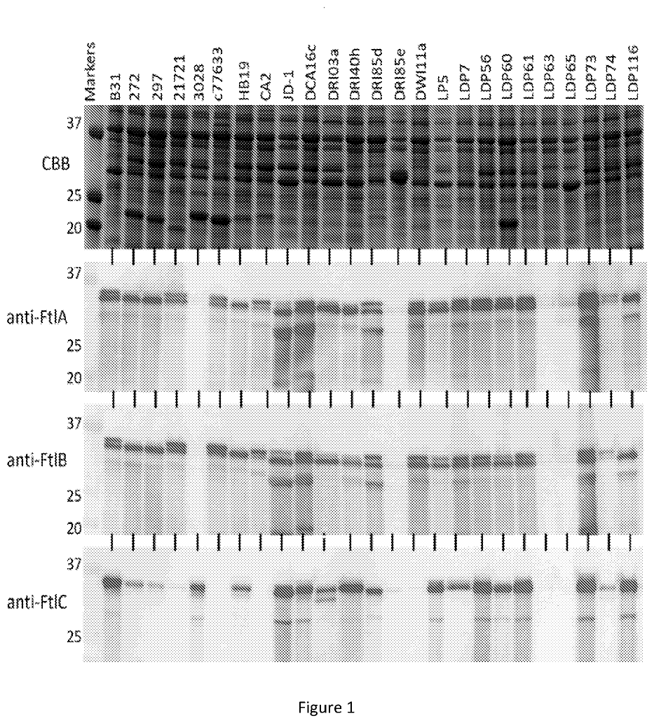

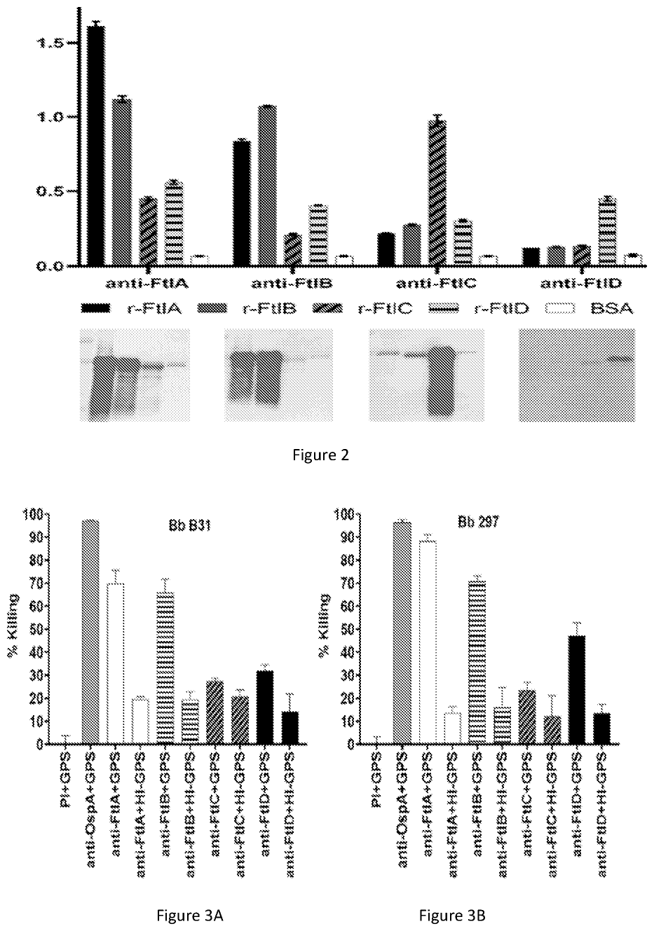

Based on the high identity between FtlA and FtlB, particularly in their N-terminal domains, we speculated that antibodies that recognize one would recognize the other. This was verified by immunoblot and ELISA analyses. Immunoblots of Borreliella cell lysates were screened with anti-FtlA, anti-FtlB, and anti-FtlC antiserum (FIG. 1). The immunoreactivity patterns were the same for FtlA and FtlB but differed for FtlC. The results were further verified using ELISA assays. The Ftl proteins were immobilized in the ELISA plate wells and on immunoblots and screened with antisera generated against each Fil protein (FIG. 2). Strong cross-reactivity of the FtlA and FtlB antisera with both FtlA and FtlB but not with FtlC and FtlD proteins was observed. Antibodies to FtlC were highly specific for FtlC, with only weak reactivity with FtlA and FtlB. The significance of this finding is that antibodies elicited by recombinant FtlA bind to both FtlA and FtlB and will therefore act synergistically to kill LD spirochete strains by binding to two protein targets on the cell surface.

To determine if anti-Ftl Abs have bactericidal activity, sera from rats immunized with FtlA, FtlB, FtlC, or FtlD were incubated with B. burgdorferi isolates B31 and 297 in the presence of complement activity-certified guinea pig serum (GPS) or heat-inactivated GPS (HI-GPS) (FIGS. 3A and B). High titer anti-OspA antiserum served as a positive control. Anti-FtlA and anti-FtlB antisera displayed potent Ab-mediated, complement-dependent bactericidal activity against both test strains. In contrast, anti-FtlC and anti-FtlD antisera had low bactericidal activity consistent with the low levels of expression of FtlC and FtlD during cultivation.

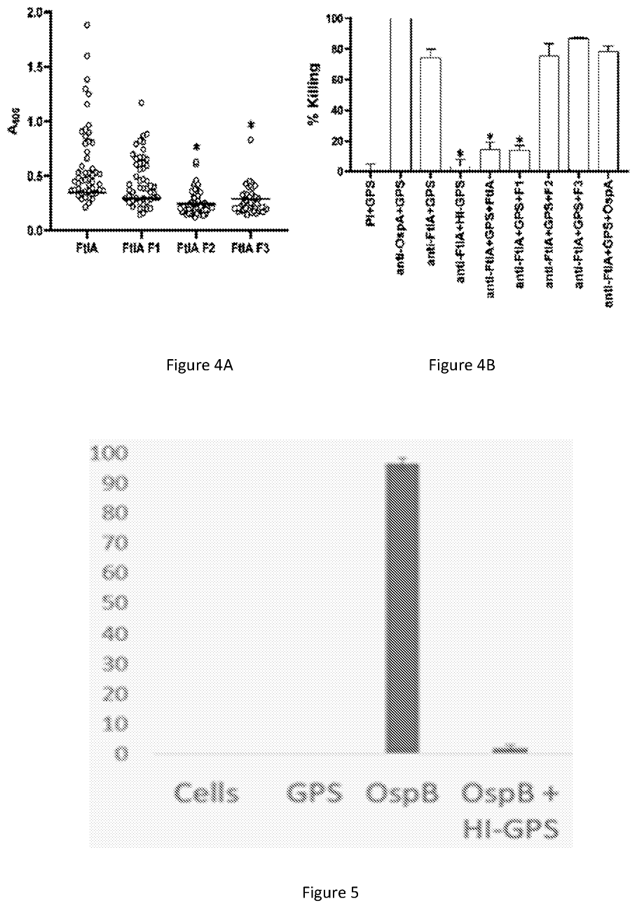

To identify the immunodominant region of FtlA, full-length FtlA (minus the leader peptide) and overlapping fragments spanning the length of the protein (F1, F2, and F3) were produced and screened with B. burgdorferi peptide C6 Ab-positive serum samples from client-owned dogs. Of the 50 dogs screened, 74% (37/50) were Ab positive for fragment F1, whereas only 44% and 28% were Ab positive for the F2 and F3 fragments, respectively (FIG. 4A). The results indicate that the immunodominant epitopes of FtlA and FtlB are localized within the N-terminal domain of each protein. To verify that the bactericidal epitope(s) of FtlA reside within its immunodominant N-terminal domain, bactericidal assays were performed in which anti-FtlA antiserum was incubated with full-length FtlA and fragments F1, F2, and F3 prior to mixing with cells and GPS (FIG. 4B). Full-length FtlA and F1 inhibited Ab-mediated killing, whereas the F2 and F3 fragments did not. As a negative control, the anti-FtlA antiserum was incubated with recombinant B. burgdorferi B31 OspA prior to mixing with cells, and as expected, OspA did not block anti-Ftl Ab-mediated complement-dependent killing. It can be concluded that the dominant epitopes that elicit bactericidal Abs are contained within the N-terminal domain of FtlA. The amino acid identity shared by the N-terminal domains of FtlA and FtlB (residues 19-143) is evident in the alignment of the N-terminal domains. Residues that differ are indicated by bolding in the sequences shown below, where the segment of FtlA is SEQ ID NO: 8 and that of FtlB is SEQ ID NO: 9.

| | |
|---|---|
| Ft1A | NLDSKLSSNKEQKNNNNVKEVSDSVQEDGLNDLYNNQEKQKSFTKNFGERKYEDLINPIEPIIPSESPKNKANIPN |
| Ft1B | NLDSKLSSNKEQKNNNNVKEVSNSVQEDGLNDLYSNQEKQKSFTKNFGEWKYEDLINPEIPIIPSESPKNKANIPN |

Due to the high degree of identity between the N-terminal domains of FtlA and FtlB, we demonstrated that only the FtlA N-terminal domain is required for inclusion in a chimeric vaccine and that antibodies to this domain will target both FtlA and FtlB and synergistically kill Borreliella species in an antibody-dependent complement-mediated killing.

### Example 2. Identification of the Immunodominant Bactericidal Epitopes of OspA and OspB

To develop an effective vaccine, it is essential to design the vaccine antigen formulation such that it induces the production of antibodies that will target the LD spirochetes in both the tick and mammal. While the FtlA protein described above is expressed in both ticks and mammals, a low percentage of isolates lack one or more FtlA proteins. Hence, a vaccine formulation that includes immunodominant domains of tick phase and mammalian phase proteins is essential. Data obtained by our lab indicated the inclusion of well-defined linear epitopes of the tick phase protein OspA and extended domains of OspB into a chimeric protein might induce antibody responses with bactericidal activity against diverse Borreliella species. The immunodominant domains of OspA and OspB were identified through epitope mapping as described above for the Ftl proteins with some modifications. Since OspA and OspB are produced selectively in ticks, animals that are naturally infected do not elicit antibody responses to these proteins. Hence, we could not use serum from naturally infected animals to identify their immunodominant epitopes. To circumvent this technical hurdle, we inoculated mice with a high dose (10⁶) of in vitro cultivated B. burgdorferi B31. OspA and OspB are produced at high levels during cultivation, and consistent with this, we were able to elicit antibodies to these proteins even though they are not produced during active infection in mammals. Another strength of this approach is that the naturally exposed domains of both proteins are presented as they are on the surface of intact cells. Serum was collected from the mice, and sub-fragments or peptides derived from each protein were screened with the sera by ELISA and immunoblotting. The OspA region spanning residues 221-240 and the OspB region spanning residues 244-260 were identified as immunodominant epitopes. Peptides corresponding to these domains induced bactericidal antibodies but only when conjugated to the carrier protein, Keyhole limpet hemocyanin (KLH). This demonstrated that the context in which the immunogenic domains are presented is a critical factor. FIG. 5 demonstrated that antibodies directed at OspB kill in a complement-dependent manner. Based on these results, we pursued a path in which a chimeric of two different OspA epitope variants (A1/A15) combined was inserted into chimeric proteins that included domains from other Borreliella proteins. Several chimerics were generated and tested, and the results are presented below.

### SEQ ID NO: 10

Protein Name: OspA (BBA15) B. burgdorferi B31

### Protein ID: AAC66260.1

NOTES: The leader peptide is omitted. The protein was readily expressed in E. coli as a soluble protein and was purified to homogeneity.

(SEQ ID NO: 10)
KQNVSSLDEKNSVSVDLPGEMKVLVSKEKNKDGKYDLIATVDKLELKGT
SDKNNGSGVLEGVKADKSKVKLTISDDLGQTTLEVFKEDGKTLVSKKVT
SKDKSSTEEKFNEKGEVSEKIITRADGTRLEYTGIKSDGSGKAKEVLKG
YVLEGTLTAEKTTLVVKEGTVTLSKNISKSGEVSVELNDTDSSAATKKT
AAWNSGISTLTITVNSKKIKDLVFTKENTITVQQYDSNGTKLEGSAVEI
TKLDEIKNALK

### SEQ ID NO: 11

### Peptide Name: A1/A15

NOTES: this chimeric peptide sequence consists of two different variants of the immunogenic domain of OspA that spans residues 221 to 240. The two epitope variants were derived from different Borreliella isolates. This epitope chimeric was not produced as a stand-alone protein but was introduced into different chimeric proteins, as detailed below, in a location that would present these epitopes on the surface of the proteins.

(SEQ ID NO: 11)
STLTITVNSKKTKDLVFTKEKTLIVSADSKKIKDFKTVFLTD

### SEQ ID NO: 12

Protein Name: OspB (BBA16) B. burgdorferi B31

### Protein ID: AAC66243.2

NOTES: The leader peptide is omitted. The protein was readily expressed in E. coli as a soluble protein and was purified to homogeneity.

(SEQ ID NO: 12)
AQKGAESIGSQKENDLNLEDSSKKSHQNAKQDLPAVTEDSVSLFNGNKI
FVSKEKNSSGKYDLRATIDQVELKGTSDKNNGSGTLEGSKPDKSKVKLT
VSADLNTVTLEAFDASNQKISSKVTKKQGSITEETLKANKLDSKKLTRS
NGTTLEYSQITDADNATKAVETLKNSIKLEGSLVGGKTTVEIKEGTVTL
KREIEKDGKVKVFLNDTAGSNKKTGKWEDSTSTLTISADSKKTKDLVFL
TDGTITVQQYNTAGTSLEGSASEIKNLSELKNALK

### SEQ ID NO: 13

### Protein Name: OspB N-Terminal Immunogenic Domain

NOTES: This polypeptide was not produced separately but was included in precursor test constructs as well as the final vaccine construct (designated as the BAF protein).

(SEQ ID NO: 13)
AQKGAESIGSQKENDLNLEDSSKKSHQNAKQDLPAVTEDSVSLFNGNKI
FVSKEKNSSGKYDLRATIDQVELKGTSDKNNGSGTLEGSKPDKSKVKLT
VSADLNTVTLEAFDASNQKISSKVTKKQGSITEETLKANKLDSKKLIRS
NGITLEYSQITDADNATKAVETLKNSIKLEGSLVGGKITVEIKEGTVTL
KREIEKDGKVKVFLNDTAGSNKKIGKWEDST

### SEQ ID NO: 14

### Protein Name: OspB C-Terminal Domain.

NOTES: This sequence is derived from the C-terminal domain of B. burgdorferi B31 OspB. It spans residues 248 to 296. This polypeptide was not produced separately but was included in precursor test constructs as well as the final BAF protein.

(SEQ ID NO: 14)
LTISADSKKIKDFVFLTDGTITVQQYNTAGTSLEGSASEIKNLSELKNA
LK

### SEQ ID NO: 27

### Peptide Name: B1/B3

NOTES: this chimeric peptide sequence consists of two different variants of the immunogenic domain of OspB that spans residues 244 to 263 (B. burgdorferi B31 numbering). The two epitope variants were derived from different Borreliella isolates. The B1 epitope is indicated by underlining. This epitope chimeric was not produced as a stand-alone protein but was introduced into different chimeric proteins, as detailed below, in a location that would present these epitopes on the surface of the proteins.

(SEQ ID NO: 27)
STLTISADSKKTKDLVFLTDNTLTVSADSKKIKDFVFLTD

### Example 3. Development of Chimeric Vaccine Antigens

Based on the data presented above, several different chimerics were generated that carried domains from two or more of the following proteins: OspC, OspA, OspB, and FtlA. The chimeric proteins, their sequences, and their properties are listed below.

### SEQ ID NO: 15

Protein Designation: OspB A1/A15 MW: 34.5 kDa Amino Acids: 320 Gene Length: 960 bp Tag: His Tag Vector: pET45
Codon optimized for expression in E. coli.

### Soluble or Insoluble Upon Expression: Soluble

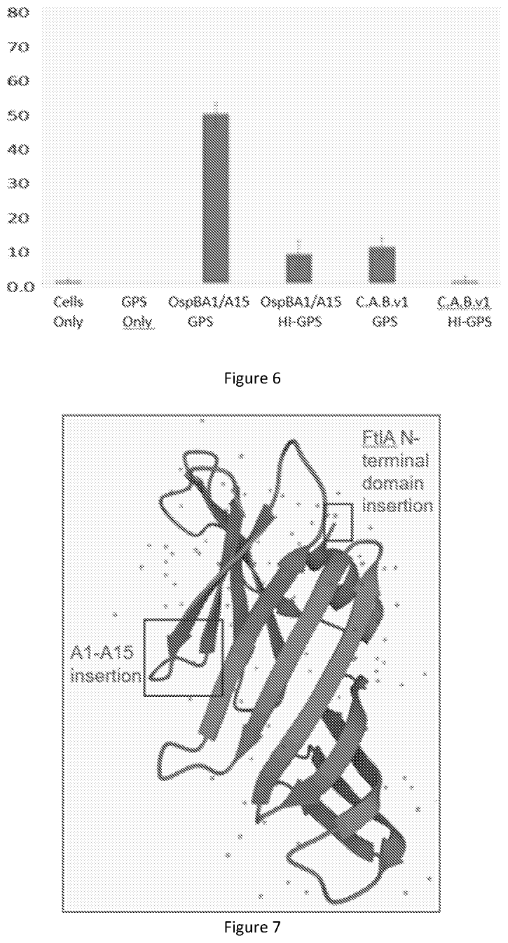

NOTES: This chimeric consists of a His tag (not shown) followed by OspB amino acids 17-243, followed by the A1/A15 OspA chimeric epitope (underlined), followed by amino acids 264-296 of OspB. The protein was produced at high levels in E. coli and was purified to homogeneity using FPLC. It was advanced to immunogenicity and protective efficacy analyses. However, while the protein was immunogenic and elicited a high titer IgG antibody response, as shown in FIG. 6, it did not elicit antibodies with significant bactericidal activity, presumably due to poor presentation of the A1/A15 domain. Hence, this chimeric was abandoned.

(SEQ ID NO: 15)
AQKGAESIGSQKENDLNLEDSSKKSHQNAKQDLPAVTEDSVSLFNGNKI
FVSKEKNSSGKYDLRATIDQVELKGTSDKNNGSGTLEGSKPDKSKVKLT
VSADLNTVTLEAFDASNQKISSKVTKKQGSITEETLKANKLDSKKLTRS
NGITLEYSQITDADNATKAVETLKNSIKLEGSLVGGKITVEIKEGTVTL
KREIEKDGKVKVFLNDTAGSNKKIGKWEDSTSTLTISADSKKTKDLVFL
TDSTLTITVNSKKTKDLVFTKEKTLIVSADSKKIKDFKIVFLIDGTITV
QQYNTAGTSLEGSASEIKNLSELKNALK

### SEQ ID NO: 16

### Protein Designation: CMT1

MW: 75.2 kDa Amino Acids: 665 Tag: His Vector: pET45. The Nucleotide Sequence was Codon Optimized for Expression in E. coli.

### Soluble or Insoluble Upon Expression: Not Applicable (See Notes Below).

Description: Chimeric consisting of his tag (not shown) followed by FtlA residues 19-143, followed by CE-BBB19 (underlined), followed by OspA amino acid residues 211-260 (italicized). The W residues that were added to increase detection sensitivity are bolded. The inclusion of two pairs of W residues increased the extinction coefficient. Extinction coefficient values were calculated using Protparam. The protein was predicted to be stable.

However, using several different methods, expression vectors, and E. coli strains, the protein was not expressed. This chimeric was abandoned, but the information obtained highlighted the importance of chimeric domain organization in determining protein properties and informed the future design of other chimerics.

(SEQ ID NO: 16)
NLDSKLSSNKEQKNNNNVKEVSDSVQEDGLNDLYNNQEKQKSFTKNFGE
RKYEDLINPIEPIIPSESPKNKANIPNISIAHTEKKETKKENLIPSTNE
EKEADAAIKYLEENILKNSKFSELIREWWSETFTNKLKEKHTDLGKEGV
TKGAEELGKLFESVEVLSKAAKEMLANSVKELTSSEEFSTKLKDNHAQL
GIQGVTKGVEELEKLSGSLESLSSEDFTKKLEGEHAQLGIENVTAAELE
KLFKAVENLAKAAKEMAKLKGEHTDLGKEGVTKGADELEKLFESVKNLS
KAAKEMLTNSKESEKFAGKLKNEHASLGKKDATKGAKELKDLSDSVESL
VKASDDFTKKLQSSHAQLGVAGGATTADELEKLFKSVESLAKAAQDALA
NSVNELTSKKLKEKHTDLGKKDATAAELEKLFESVENLAKAAKEMLSNS
NKAFTDKLKSSHAELGIANGAATKGAQELEKLFESVKNLSKAAQETLNN
SVKESESFTSEKFTKKLSESHADIGIQALKTNPTKTKGAEELDKLFKAV
ENLSKAAKEMLANSSEDFTNKLKNGNAQLGLAALKTNGTNDKGAKELKD
LSDSVESLVKAAQVMLTNSVKELTSWWKKTAAWNSGTSTLTITVNSKKT
KDLVFTKENTITVQQYDSNGTKLEGSAV

### SEQ ID NO: 17

### Protein Name: CMT2

MW: 69.8 kDa Amino Acids: 615 Tag: His Vector: pET45b (+). The Nucleotide Sequence was Codon Optimized for Expression in E. coli.
Description: Chimeric consisting of his tag (not shown) followed by FtlA residues 19-143 followed by CE-BBB19 (underlined). As described above, W residues (bolded) were added to allow for increased detection sensitivity.
Important note: In spite of predictive analyses that suggested this protein could be readily expressed, stable, and present the desired immunogenic domains on its surface, we were unable to achieve sufficient expression in E. coli. It was clear from the research done on this chimeric that predictive algorithms are of limited value. This chimeric was abandoned, but the information obtained highlighted the importance of chimeric domain organization and informed the future design of other chimerics.

(SEQ ID NO: 17)
NLDSKLSSNKEQKNNNNVKEVSDSVQEDGLNDLYNNQEKQKSFTKNFGE
RKYEDLINPIEPIIPSESPKNKANIPNISIAHTEKKETKKENLIPSTNE
EKEADAAIKYLEENILKNSKFSELIREWWSETFTNKLKEKHTDLGKEGV
TKGAEELGKLFESVEVLSKAAKEMLANSVKELTSSEEFSTKLKDNHAQL
GIQGVTKGVEELEKLSGSLESLSSEDFTKKLEGEHAQLGIENVTAAELE
KLFKAVENLAKAAKEMAKLKGEHTDLGKEGVTKGADELEKLFESVKNLS
KAAKEMLTNSKESEKFAGKLKNEHASLGKKDATKGAKELKDLSDSVESL
VKASDDFTKKLQSSHAQLGVAGGATTADELEKLFKSVESLAKAAQDALA
NSVNELTSKKLKEKHTDLGKKDATAAELEKLFESVENLAKAAKEMLSNS
NKAFTDKLKSSHAELGIANGAATKGAQELEKLFESVKNLSKAAQETLNN
SVKESESFTSEKFTKKLSESHADIGIQALKTNPTKTKGAEELDKLFKAV
ENLSKAAKEMLANSSEDFTNKLKNGNAQLGLAALKTNGTNDKGAKELKD
LSDSVESLVKAAQVMLTNSVKELTSWW

### SEQ ID NO: 18

### Protein Name: CMT3

MW: 84.5 kDa Amino Acids: 742 Tag: His Vector: pET45b (+); The Nucleotide Sequence was Codon Optimized for Expression in E. coli
Description: Chimeric consisting of His tag (not shown) followed by FtlA residues 19-143 (italicized), followed by two W residues (bold), followed by CE-BBB19, followed by two W residues (bold), followed by a second copy of FtlA residues 19-143 (italicized and underlined) followed by two W residues (bold). As detailed above, all added W residues were introduced to increase detection sensitivity.
Notes: Several induction methods, strains, and plasmid vectors were tested to induce the production of CMT3 in E. coli. None resulted in sufficient expression. Hence, the chimeric was abandoned. However, the information obtained in the analysis of CMT3 provided insight into the importance of epitope/domain organization and informed the design of the final vaccine formulation chimerics.

(SEQ ID NO: 18)
NLDSKLSSNKEQKNNNNVKEVSDSVQEDGLNDLYNNQEKQKSFTKNFGE
RKYEDLINPIEPIIPSESPKNKANIPNISIAHTEKKETKKENLIPSTNE
EKEADAAIKYLEENILKNSKFSELIREWWSETFTNKLKEKHTDLGKEGV
TKGAEELGKLFESVEVLSKAAKEMLANSVKELTSSEEFSTKLKDNHAQL
GIQGVTKGVEELEKLSGSLESLSSEDFTKKLEGEHAQLGIENVTAAELE
KLFKAVENLAKAAKEMAKLKGEHTDLGKEGVTKGADELEKLFESVKNLS
KAAKEMLTNSKESEKFAGKLKNEHASLGKKDATKGAKELKDLSDSVESL
VKASDDFTKKLQSSHAQLGVAGGATTADELEKLFKSVESLAKAAQDALA
NSVNELTSKKLKEKHTDLGKKDATAAELEKLFESVENLAKAAKEMLSNS
NKAFTDKLKSSHAELGIANGAATKGAQELEKLFESVKNLSKAAQETLNN
SVKESESFTSEKFTKKLSESHADIGIQALKTNPTKTKGAEELDKLFKAV
ENLSKAAKEMLANSSEDFTNKLKNGNAQLGLAALKTNGTNDKGAKELKD
LSDSVESLVKAAQVMLTNSVKELISWWNLDSKLSSNKEQKNNNNVKEVS
DSVQEDGLNDLYNNQEKQKSFTKNFGERKYEDLINPIEPIIPSESPKNK
ANIPNISIAHTEKKETKKENLIPSTNEEKEADAAIKYLEENILKNSKFS
ELIREWW

### SEQ ID NO: 19

### Gene Name: C.A.B.v1

MW: 48.9 kDa Amino Acids: 452 Gene Length: Tag: His Tag Vector: pET45b
Codon Optimized or Wild-Type: E. coli Optimized

### Soluble or Insoluble Upon Expression: Soluble.

Description: The chimeric consists of a His tag (not shown) followed by CE-BBB19, followed by A1/A15 chimeric epitopic (underlined), followed by the OspB chimeric epitope, B1/B3 (italicized).
NOTES: As shown in FIG. 6, while the C.A.B.v1 construct was immunogenic in mice, it failed to induce antibodies with bactericidal activity. These findings indicate that the context in which the component epitopes are presented defines protective efficacy. This construct was not pursued.

Amino acid sequence:
(SEQ ID NO: 19)
SETFTNKLKEKHTDLGKEGVTKGAEELGKLFESVEVLSKAAKEMLANSV
KELTSSEEFSTKLKDNHAQLGIQGVTKGVEELEKLSGSLESLSSEDFTK
KLEGEHAQLGIENVTAAELEKLFKAVENLAKAAKEMAKLKGEHTDLGKE
GVTKGADELEKLFESVKNLSKAAKEMLTNSKESEKFAGKLKNEHASLGK
KDATKGAKELKDLSDSVESLVKASDDFTKKLQSSHAQLGVAGGATTADE
LEKLFKSVESLAKAAQDALANSVNELTSKKLKEKHTDLGKKDATAAELE
KLFESVENLAKAAKEMLSNSNKAFTDKLKSSHAELGIANGAATKGAQEL
EKLFESVKNLSKAAQETLNNSVKESESFTSILTITVNSKKTKDLVFTKE
KTLIVSADSKKIKDFKTVFLTDSTLTISADSKKTKDLVFLTDNTLTVSA
DSKKIKDFVFLTD

### SEQ ID NO: 1

Gene name: BAF
MW: 52.2 kDa; PI: 6.76 Amino Acids: 449; Tag: His; Vector: pET45. The Nucleotide Sequence was Codon Optimized for Expression in E. coli Optimized. BAF was Soluble Upon Expression in E. coli.
Description: Consists of a His tag (not shown), followed by OspB residues 17-243, followed by A1/A15 chimeric OspA epitopic (underlined and italicized), followed by residues OspB 244-296, followed by two tryptophan residues followed by residues 19-143 of FtlB, followed by two W resides. The structural organization of BAF is depicted in FIG. 7. The two pairs of W residues were added to increase detection sensitivity. The inclusion of the two pairs of W residues changed the extinction coefficient from 14440 (Abs 0.1%=0.295) to 36440 (Abs 0.1% (1 g/L)=0.734). Extinction coefficients are in units of M⁻¹ cm⁻¹ at 280 nm measured in water. Values were calculated using Protparam. The introduction of the W residues had no significant impact on the calculated instability index, which was approximately 29. A value of 29 is indicative of a stable protein.

BAF Protein sequence
(SEQ ID NO: 1)
AQKGAESIGSQKENDLNLEDSSKKSHQNAKQDLPAVTEDSVSLFNGNKI
FVSKEKNSSGKYDLRATIDQVELKGTSDKNNGSGTLEGSKPDKSKVKLT
VSADLNTVTLEAFDASNQKISSKVTKKQGSITEETLKANKLDSKKLTRS
NGITLEYSQITDADNATKAVETLKNSIKLEGSLVGGKTTVEIKEGTVTL
KREIEKDGKVKVFLNDTAGSNKKTGKWEDSTSTLTISADSKKTKDLVFL
TDSTLTITVNSKKTKDLVFTKEKTLTVSADSKKIKDFVFLTDGTITVQQ
YNTAGTSLEGSASEIKNLSELKNALKWWNLDSKLSSNKEQKNNNNVKEV
SDSVQEDGLNDLYNNQEKQKSFTKNFGERKYEDLINPIEPIIPSESPKN
KANIPNISIAHTEKKETKKENLIPSTNEEKEADAAIKYLEENILKNSKF
SELIREWW

Exemplary BAF DNA sequence: note that the sequence was codon optimized for expression in E. coli. Any nucleotide sequence that encodes the protein sequence above is suitable for protein production.

(SEQ ID NO: 20)
GCGCAGAAGGGTGCGGAGAGCATCGGCAGCCAAAAAGAAAACGACCTGA
ACCTGGAGGATAGCAGCAAGAAAAGCCACCAGAACGCGAAGCAAGACCT
GCCGGCGGTGACCGAGGATAGCGTTAGCCTGTTCAACGGTAACAAGATC
TTTGTTAGCAAGGAGAAGAACAGCAGCGGCAAATACGACCTGCGTGCGA
CCATTGATCAGGTGGAGCTGAAGGGTACCAGCGACAAAAACAACGGTAG
CGGCACCCTGGAAGGCAGCAAACCGGATAAGAGCAAAGTGAAGCTGACC
GTTAGCGCGGACCTGAACACCGTTACCCTGGAGGCGTTTGACGCGAGCA
ACCAGAAGATCAGCAGCAAAGTGACCAAGAAACAAGGTAGCATTACCGA
GGAAACCCTGAAGGCGAACAAACTGGACAGCAAGAAACTGACCCGTAGC
AACGGCACCACCCTGGAATATAGCCAAATCACCGACGCGGATAACGCGA
CCAAGGCGGTTGAGACCCTGAAGAACAGCATTAAACTGGAAGGTAGCCT
GGTGGGTGGCAAAACCACCGTTGAGATCAAAGAAGGTACCGTGACCCTG
AAGCGTGAGATTGAAAAAGACGGCAAAGTGAAGGTTTTTCTGAACGATA
CCGCGGGTAGCAACAAGAAAACCGGCAAGTGGGAGGACAGCACCAGCAC
CCTGACCATCAGCGCGGACAGCAAGAAAACCAAAGATCTGGTTTTCCTG
ACCGACAGCACCCTGACCATTACCGTGAACAGCAAGAAAACCAAGGATC
TGGTTTTTACCAAAGAAAAGACCCTGACCGTGAGCGCGGATAGCAAGAA
AATCAAAGACTTCGTTTTTCTGACCGATGGTACCATTACCGTGCAGCAA
TACAACACCGCGGGTACCAGCCTGGAAGGCAGCGCGAGCGAGATCAAAA
ACCTGAGCGAGCTGAAGAACGCGCTGAAATGGTGGAACCTGGACAGCAA
GCTGAGCAGCAACAAGGAACAGAAAAACAACAACAACGTGAAAGAAGTT
AGCGATAGCGTGCAAGAGGACGGTCTGAACGATCTGTACAACAACCAGG
AGAAACAAAAGAGCTTCACCAAGAACTTTGGCGAGCGTAAATATGAAGA
CCTGATCAACCCGATTGAACCGATCATTCCGAGCGAGAGCCCGAAAAAC
AAGGCGAACATCCCGAACATCAGCATTGCGCACACCGAGAAGAAAGAAA
CCAAGAAAGAGAACCTGATTCCGAGCACCAACGAGGAAAAGGAAGCGGA
CGCGGCGATCAAATATCTGGAGGAAAACATTCTGAAGAACAGCAAATTC
AGCGAGCTGATTCGTGAATGGTGG

Important notes: BAF can be reliably expressed at high levels in E. coli and purified to homogeneity by FPLC with high yield. BAF was advanced for further evaluation, including immunogenicity analyses, and the ability to induce protective immunity in mice, rats and Rhesus macaques. BAF is one of the two chimerics in the vaccine formulation. Data demonstrating the protective efficacy of the BAF/CE-BBB19 formulation are detailed below. The BAK construct is designed to elicit antibodies that target FtlA, FtlB, OspA, and OspB. This is a defining aspect of this novel vaccine antigen and represents a significant advancement in the art. The linkage of several epitopes derived from several different proteins is a significant advancement.

### SEQ ID NO: 21

Gene Name: OspC (type A)

### Protein ID: AAC66329

NOTES: We have identified two immunodominant epitopes in the Borreliella OspC protein designated as L5 (loop 5) and H5 (helix 5). CE-BBB19 was designed to include L5H5 chimeric epitopes from 10 distinctly different OspC types derived from North American Borreliella OspC proteins. Although the total number of amino acids varies among OspC types, here we use the amino acid numbering assigned to B. burgdorferi B31 OspC type A sequence. Note that the leader peptide was omitted. The L5 epitope spans amino acids 136 to 150, and the H5 epitope spans 168 to 203. The region of the OspC that contains the L5 and H5 epitopes is indicated by underlining and italicizing, respectively.

Protein sequence:
(SEQ ID NO: 21)
NNSGKDGNTSANSADESVKGPNLTEISKKITDSNAVLLAVKEVEALLSS
IDEIAAKAIGKKIHQNNGLDTENNHNGSLLAGAYAISTLIKQKLDGLKN
EGLKEKIDAAKKCSETFTNKLKEKHTDLGKEGVTDADAKEAILKTNGTK
TKGAEELGKLFESVEVLSKAAKEMLANSVKELTSPVVAESPKKP

### SEQ ID NO: 2

### Gene Name and Alternative Names: CE-BBB19

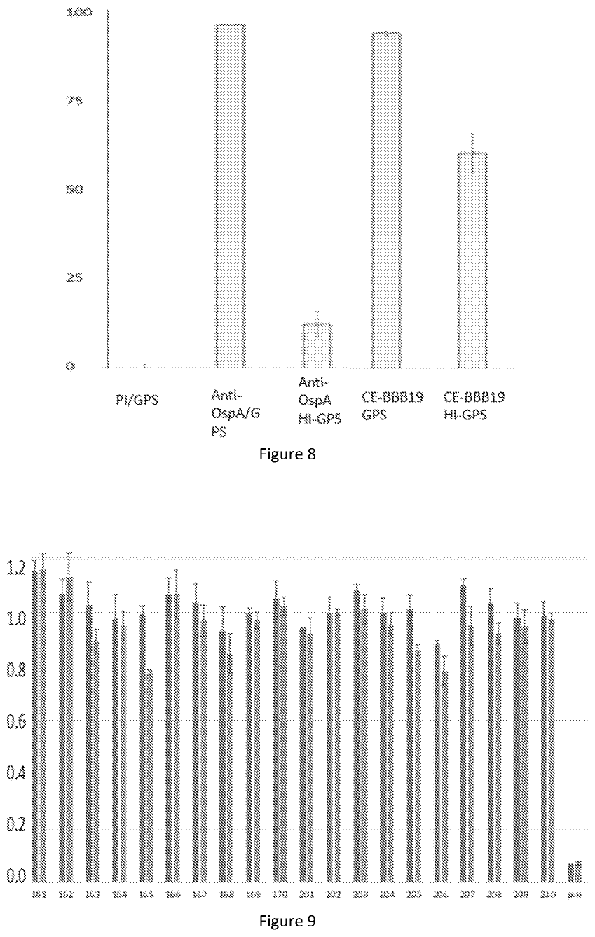

MW: 52.18 kDa; Tag: His (not shown), Amino Acids: 485, Vector: pET45. The Coding Sequence was Codon Optimized for Expression in E. coli.
Description: Consists of L5 and H5 epitope chimeras from OspC types that are associated with human infection. Below, the sequence is presented in contiguous form. NOTES: Antiserum raised against CE-BBB19 binds to a diverse array of OspC types (data not shown). The antibodies elicited by immunization of rats and mice also displayed potent bactericidal activity (FIG. 8). Strikingly, the antibody was able to elicit significant killing in a complement-independent manner as well. This indicates that antibody binding is blocking a critical activity of OspC.

CE-BBB19 amino acid sequence:
(SEQ ID NO: 2)
SETFTNKLKEKHTDLGKEGVTKGAEELGKLFESVEVLSKAAKEMLANSV
KELTSSEEFSTKLKDNHAQLGIQGVTKGVEELEKLSGSLESLSSEDFTK
KLEGEHAQLGIENVTAAELEKLFKAVENLAKAAKEMAKLKGEHTDLGKE
GVIKGADELEKLFESVKNLSKAAKEMLTNSKESEKFAGKLKNEHASLGK
KDATKGAKELKDLSDSVESLVKASDDFTKKLQSSHAQLGVAGGATTADE
LEKLFKSVESLAKAAQDALANSVNELTSKKLKEKHTDLGKKDATAAELE
KLFESVENLAKAAKEMLSNSNKAFTDKLKSSHAELGIANGAATKGAQEL
EKLFESVKNLSKAAQETLNNSVKESESFTSEKFTKKLSESHADIGIQAL
KTNPTKTKGAEELDKLFKAVENLSKAAKEMLANSSEDFTNKLKNGNAQL
GLAALKTNGTNDKGAKELKDLSDSVESLVKAAQVMLTNSVKELTS

Below is the CE-BBB19 amino acid sequence shown above. In this version, the L5 (underlined) and H5 (italicized) epitope sequences derived from each OspC type are indicated. Note that the lengths of the epitopes from each OspC type differ. The reason for this is that in designing the construct, amino residues that were repeated were trimmed from some epitopes. This allowed us to reduce the overall length of the protein, which enhanced protein production in E. coli:

CE-BBB19 amino acid sequence (SEQ ID NO: 2) with epitopes from each OspC
type delineated :
/------------------OspC type A--------------------------/------OspC type B---
SETFTNKLKEKHTDLGKEGVTKGAEELGKLFESVEVLSKAAKEMLANSVKELTSSEEFSTKLKDNHAQLGIQGVTKG
-----------------/------------OspC type K-----------------/----OspC type I---
VEELEKLSGSLESLSSEDFTKKLEGEHAQLGIENVTAAELEKLFKAVENLAKAAKEMAKLKGEHTDLGKEGVTKGAD
-------------------------/-----------OspC type H------------------/----------
ELEKLFESVKNLSKAAKEMLTNSKESEKFAGKLKNEHASLGKKDATKGAKELKDLSDSVESLVKASDDFTKKLQSSH
---OspC type N---------------------------/------------OspC type C------------
AQLGVAGGATTADELEKLFKSVESLAKAAQDALANSVNELTSKKLKEKHTDLGKKDATAAELEKLFESVENLAKAAK
-------/-------------OspC type M--------------------------/--------------OspC
EMLSNSNKAFTDKLKSSHAELGIANGAATKGAQELEKLFESVKNLSKAAQETLNNSVKESESFTSEKFTKKLSESHA
Type U----------------------------------/----------OspC type F---------------
DIGIQALKTNPTKTKGAEELDKLFKAVENLSKAAKEMLANSSEDFTNKLKNGNAQLGLAALKTNGTNDKGAKELKDL
------------------------/
SDSVESLVKAAQVMLTNSVKELTS

Exemplary CE-BBB19 DNA sequence: Note that the CE-BBB19 sequence was codon optimized for expression in E. coli and thus does not match the wild-type natural sequence of each epitope. Importantly, any nucleotide sequence that encodes the protein sequence above is suitable for protein production.

(SEQ ID NO: 22)
AGCGAGACCTTTACCAACAAACTGAAGGAGAAACACACCGATCTGGGCA
AGGAAGGCGTTACCAAGGGTGCGGAGGAACTGGGCAAGCTGTTCGAGAG
CGTGGAAGTTCTGAGCAAGGCGGCGAAAGAGATGCTGGCGAACAGCGTG
AAAGAACTGACCAGCAGCGAGGAATTTAGCACCAAGCTGAAAGACAACC
ACGCGCAGCTGGGTATCCAAGGCGTGACCAAGGGTGTTGAGGAACTGGA
GAAACTGAGCGGCAGCCTGGAAAGCCTGAGCAGCGAGGATTTCACCAAG
AAACTGGAGGGTGAACACGCGCAGCTGGGCATTGAGAACGTGACCGCGG
CGGAGCTGGAAAAGCTGTTTAAAGCGGTGGAAAACCTGGCGAAGGCGGC
GAAAGAAATGGCGAAGCTGAAAGGCGAGCACACCGACTTAGGCAAGGAA
GGCGTTACTAAAGGCGCGGATGAGCTGGAAAAACTGTTTGAGAGCGTGA
AAAACCTGAGCAAAGCGGCGAAGGAAATGCTGACCAACAGCAAGGAGAG
CGAAAAATTTGCGGGCAAGCTGAAAAACGAGCACGCGAGCCTGGGTAAG
AAAGACGCGACCAAGGGCGCGAAAGAGCTGAAGGACCTGAGCGATAGCG
TGGAAAGCCTGGTTAAGGCGAGCGACGATTTCACCAAGAAATTACAGAG
CAGCCACGCGCAACTGGGTGTTGCGGGTGGCGCGACCACCGCGGACGAA
CTGGAGAAACTGTTTAAGAGCGTTGAAAGCCTGGCGAAAGCGGCGCAGG
ATGCGCTGGCGAATAGCGTTAACGAGCTGACCAGCAAGAAACTGAAGGA
AAAACACACCGACCTGGGCAAGAAAGATGCTACCGCGGCGGAGTTAGAG
AAACTGTTTGAGAGCGTGGAAAACCTGGCGAAAGCGGCGAAGGAGATGC
TGAGCAACAGCAACAAAGCGTTTACCGACAAGCTGAAAAGCAGCCACGC
GGAACTGGGTATCGCGAACGGTGCGGCGACCAAGGGTGCGCAGGAGCTG
GAAAAACTGTTCGAGAGCGTTAAGAATCTGAGCAAGGCGGCGCAAGAAA
CCCTGAACAACAGCGTGAAGGAGAGCGAAAGCTTCACCAGCGAGAAATT
TACCAAGAAACTGAGCGAAAGCCACGCGGACATCGGTATTCAGGCGCTG
AAGACCAACCCGACCAAGACCAAAGGCGCGGAGGAACTGGATAAACTGT
TCAAGGCGGTTGAGAATCTGAGCAAAGCGGCGAAAGAAATGCTGGCGAA
CAGCAGCGAAGACTTTACCAACAAGCTGAAAAACGGTAACGCGCAGCTG
GGTCTGGCGGCGCTGAAAACCAACGGTACCAACGATAAAGGCGCGAAGG
AGCTGAAAGATCTGAGCGACAGCGTTGAGAGCCTGGTTAAGGCGGCGCA
AGTGATGCTGACCAACAGCGTTAAAGAACTGACCAGC

### Example 4. Coadministration of BAF and CE-BBB19 Elicits Strong Bactericidal Antibody Responses with an IgG1 Skew

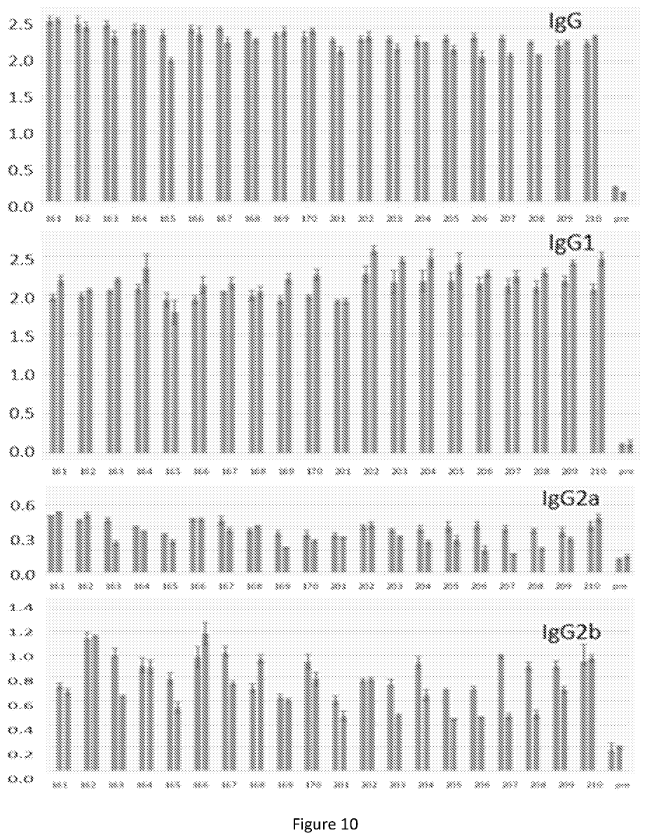

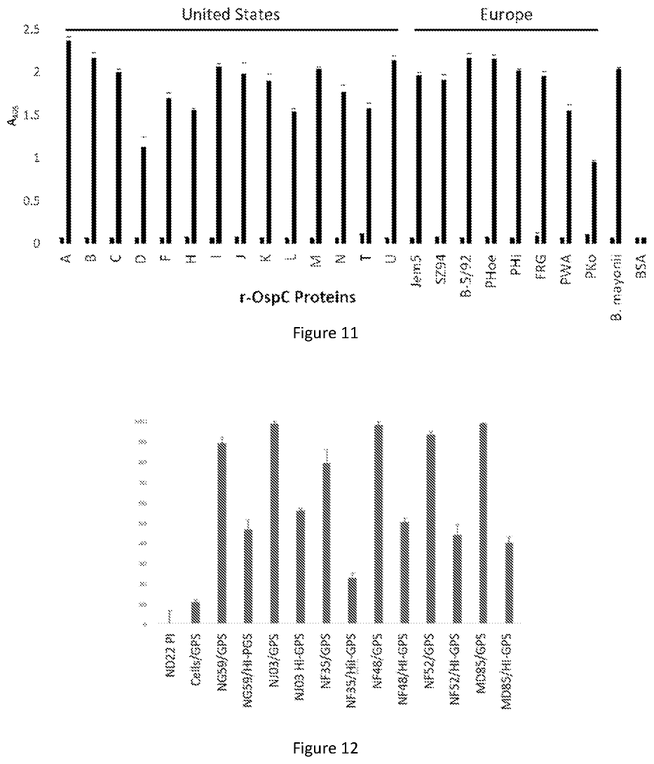

To determine if BAF and CE-BBB19 elicit synergistic immunogenicity, equimolar amounts of each protein were combined and used to immunize mice and rats. When delivered together, both proteins elicited high IgG titers (FIG. 9). IgG isotyping analyses revealed a skew to IgG1 skew (FIG. 10). A dominant IgG1 response is important because IgG1 is the most efficient IgG subtype at complement fixation. Delivery of both antigens together also resulted in a broad response to diverse OspC types of North American and European origin, including the OspC protein of Borreliella mayonii, an emerging species in North America (FIG. 11).

### Example 6. Demonstration of Vaccine Efficacy in a Rhesus Macaque Non-Human Primate LD Model

Non-human primate models represent the gold standard system for assessing immunogenicity and protective efficacy of vaccines designed for use in humans. Six animals were immunized with two doses of BAF/CE-BBB19 in alum. Control animals received two doses of alum alone. Serum samples were collected prior to immunization and three weeks after the second vaccine dose. After collecting the post-vaccination serum samples, the animals were challenged with Ixodes scapularis ticks infected with B. burgdorferi B31. The ticks were fed to repletion. Serum from the pre-challenged animals was tested for antibody levels to the vaccine antigens and for bactericidal activity. Immunization induced high antibody titers (data not shown). Bactericidal assays demonstrated that the BAF/CE-BBB19 formulation elicits potent complement-dependent and complement-independent bactericidal activity (FIG. 12). This important finding demonstrates that killing occurs through two synergistic mechanisms. This is a novel finding that allows for greater vaccine efficacy.

While the invention has been described in terms of its several exemplary embodiments, those skilled in the art will recognize that the invention can be practiced with modification within the spirit and scope of the appended claims. Accordingly, the present invention should not be limited to the embodiments as described above but should further include all modifications and equivalents thereof within the spirit and scope of the description provided herein.

1. A recombinant chimeric protein having
i) an amino acid sequence as set forth in SEQ ID NO: 1, or
ii) an amino acid sequence that has at least 95% identity to the amino acid sequence as set forth in SEQ ID NO: 1, or
iii) an amino acid sequence as set forth in SEQ ID NO: 1 but from which one or more groups of 1-10 contiguous amino acids have been removed at one or more positions within SEQ ID NO. 1.

2. The recombinant chimeric protein of claim 1 wherein the one or more groups of 1-10 contiguous amino acids correspond to one or more epitopes included in the recombinant chimeric protein.

3. A recombinant chimeric protein having
i) an amino acid sequence as set forth in SEQ ID NO: 2, or
ii) an amino acid sequence that has at least 95% identity to the amino acid sequence as set forth in SEQ ID NO: 2, or
iii) an amino acid sequence as set forth in SEQ ID NO: 2 but from which one or more groups of 1-10 contiguous amino acids have been removed at one or more positions within SEQ ID NO. 2.

4. The recombinant chimeric protein of claim 3 wherein the one or more groups of 1-10 contiguous amino acids correspond to one or more epitopes included in the recombinant chimeric protein.

5. A composition comprising
the recombinant chimeric protein of claim 1;
and
a physiologically acceptable carrier.

6. The composition of claim 5, further comprising an adjuvant.

7. A lyophilized, reconstitutable preparation comprising the recombinant protein of claim 1.

8. A nucleic acid encoding the recombinant chimeric polypeptide of claim 1.

9. A nucleic acid encoding the recombinant chimeric polypeptide of claim 3.

10. A vector comprising the nucleic acid of claim 9.

11. A host cell comprising the vector of claim 10.

12. An Escherichia coli bacterial cell that is genetically engineered to contain and express the nucleic acid of claim 9.

13. A method of vaccinating a mammal against Lyme disease caused by Borreliella, comprising,
administering to the mammal a therapeutically effective dose of the composition of claim 5, wherein the therapeutically effective dose elicits an immune response against the Borreliella.

14. The method of claim 13, wherein the immune response includes both an antibody-mediated complement-dependent response and a complement-independent response.

15. The method of claim 13, wherein the immune response is a protective immune response.

16. The method of claim 13, wherein the mammal is a human or a non-human mammal.

17. The method of claim 13, wherein the Borreliella is B. burgdorferi, B, garinii, B. mayonii, B. afzelii, B. carolinesis, B. americanum, B. bavariensis, B. andersonii, B. japonica, B. spielmanii, B. sinica, B. kurtenbachii, B. baltazardii and/or B. venezuelensis.

18. A composition comprising
the recombinant chimeric protein of claim 2; and
a physiologically acceptable carrier.

19. A composition comprising
the recombinant chimeric protein of claim 3; and
a physiologically acceptable carrier.

20. A composition comprising
the recombinant chimeric protein of claim 4; and
a physiologically acceptable carrier.

21. A nucleic acid encoding the recombinant chimeric polypeptide of claim 2.

22. A vector comprising the nucleic acid of claim 22.

23. A host cell comprising the vector of claim 22.

24. The host cell of claim 23 wherein the host cell is a genetically engineered Escherichia coli bacterial cell that is genetically engineered to contain and express the nucleic acid on the vector.

25. A vector comprising the nucleic acid of claim 8.

26. A host cell comprising the vector of claim 25.

27. The host cell of claim 26 wherein the host cell is a genetically engineered Escherichia coli bacterial cell that is genetically engineered to contain and express the nucleic acid on the vector.

28. A nucleic acid encoding the recombinant chimeric polypeptide of claim 4.

29. A vector comprising the nucleic acid of claim 28.

30. A host cell comprising the vector of claim 29.

31. The host cell of claim 30 wherein the host cell is a genetically engineered Escherichia coli bacterial cell that is genetically engineered to contain and express the nucleic acid on the vector.

32. A method of vaccinating a mammal against Lyme disease caused by Borreliella, comprising,
administering to the mammal a therapeutically effective dose of the composition of claim 18, wherein the therapeutically effective dose elicits an immune response against the Borreliella.

33. The method of claim 32, wherein the immune response includes both an antibody-mediated complement-dependent response and a complement-independent response.

34. The method of claim 32, wherein the immune response is a protective immune response.

35. The method of claim 32, wherein the mammal is a human or a non-human mammal.

36. The method of claim 32, wherein the Borreliella is B. burgdorferi, B, garinii, B. mayonii, B. afzelii, B. carolinesis, B. americanum, B. bavariensis, B. andersonii, B. japonica, B. spielmanii, B. sinica, B. kurtenbachii, B. baltazardii and/or B. venezuelensis.

37. A method of vaccinating a mammal against Lyme disease caused by Borreliella, comprising,
administering to the mammal a therapeutically effective dose of the composition of claim 19, wherein the therapeutically effective dose elicits an immune response against the Borreliella.

38. The method of claim 37, wherein the immune response includes both an antibody-mediated complement-dependent response and a complement-independent response.

39. The method of claim 37, wherein the immune response is a protective immune response.

40. The method of claim 37, wherein the mammal is a human or a non-human mammal.

41. The method of claim 37, wherein the Borreliella is B. burgdorferi, B, garinii, B. mayonii, B. afzelii, B. carolinesis, B. americanum, B. bavariensis, B. andersonii, B. japonica, B. spielmanii, B. sinica, B. kurtenbachii, B. baltazardii and/or B. venezuelensis.

42. A method of vaccinating a mammal against Lyme disease caused by Borreliella, comprising,
administering to the mammal a therapeutically effective dose of the composition of claim 20, wherein the therapeutically effective dose elicits an immune response against the Borreliella.

43. The method of claim 42, wherein the immune response includes both an antibody-mediated complement-dependent response and a complement-independent response.

44. The method of claim 42, wherein the immune response is a protective immune response.

45. The method of claim 42, wherein the mammal is a human or a non-human mammal.

46. The method of claim 42, wherein the Borreliella is B. burgdorferi, B, garinii, B. mayonii, B. afzelii, B. carolinesis, B. americanum, B. bavariensis, B. andersonii, B. japonica, B. spielmanii, B. sinica, B. kurtenbachii, B. baltazardii and/or B. venezuelensis.

47. A lyophilized, reconstitutable preparation comprising the recombinant protein of claim 2.

48. A lyophilized, reconstitutable preparation comprising the recombinant protein of claim 3.

49. A lyophilized, reconstitutable preparation comprising the recombinant protein of claim 4.
